SEQUENCE LISTING (28 sequences)

SEQ ID NO: 1 - DNA, 20 bp, Artificial sequence
  Synthetic primer
ggcagggacttgatagacga

SEQ ID NO: 2 - DNA, 20 bp, Artificial sequence
  Synthetic primer
gcagctcttgcacgtcatac

SEQ ID NO: 3 - DNA, 19 bp, Artificial sequence
  Synthetic primer
agctgagcgcttctgctct

SEQ ID NO: 4 - DNA, 22 bp, Artificial sequence
  Synthetic primer
agctctcatgtaccaccatgaa

SEQ ID NO: 5 - DNA, 20 bp, Artificial sequence
  Synthetic primer
gggcaccttcttgcgtttat

SEQ ID NO: 6 - DNA, 24 bp, Artificial sequence
  Synthetic primer
tcattgaaccaaagagtaacctca

SEQ ID NO: 7 - DNA, 20 bp, Artificial sequence
  Synthetic primer
aaagtctgatgccaatgcaa

SEQ ID NO: 8 - DNA, 20 bp, Artificial sequence
  Synthetic primer
gcatccacattggcttctct

SEQ ID NO: 9 - DNA, 22 bp, Artificial sequence
  Synthetic primer
tccggaaataggtcttctgtgt

SEQ ID NO: 10 - DNA, 20 bp, Artificial sequence
  Synthetic primer
cggagctgattttcctatgg

SEQ ID NO: 11 - DNA, 20 bp, Artificial sequence
  Synthetic primer
cgacagcgagtgacacaaat

SEQ ID NO: 12 - DNA, 20 bp, Artificial sequence
  Synthetic primer
atccattgcattctgcacct

SEQ ID NO: 13 - DNA, 20 bp, Artificial sequence
  Synthetic primer
gacgagatgatccccttcaa

SEQ ID NO: 14 - DNA, 20 bp, Artificial sequence
  Synthetic primer
agggctcctgagaggtttgt

SEQ ID NO: 15 - DNA, 20 bp, Artificial sequence
  Synthetic primer
tggtggctcctgtaactggt

SEQ ID NO: 16 - DNA, 20 bp, Artificial sequence
  Synthetic primer
agagctccatcctgcacagt

SEQ ID NO: 17 - DNA, 20 bp, Artificial sequence
  Synthetic primer
gtatgggatccgctgtcaac

SEQ ID NO: 18 - DNA, 20 bp, Artificial sequence
  Synthetic primer
aaggagtcggaggagcagac

SEQ ID NO: 19 - DNA, 20 bp, Artificial sequence
  Synthetic primer
tggctcaaggaactctcctg

SEQ ID NO: 20 - DNA, 20 bp, Artificial sequence
  Synthetic primer
ataactctgcgctcgttcgt

SEQ ID NO: 21 - DNA, 21 bp, Artificial sequence
  Synthetic primer
taggcgcgagctaagcaggag

SEQ ID NO: 22 - DNA, 25 bp, Artificial sequence
  Synthetic primer
gtaggcacactcaaacaacgactgg

SEQ ID NO: 23 - DNA, 20 bp, Artificial sequence
  Synthetic primer
ttgctcacaaaggtcacagg

SEQ ID NO: 24 - DNA, 20 bp, Artificial sequence
  Synthetic primer
cgtcccacctagaatctgct

SEQ ID NO: 25 - DNA, 21 bp, Artificial sequence
  Synthetic primer
ccccacacacatgcacttacc

SEQ ID NO: 26 - DNA, 21 bp, Artificial sequence
  Synthetic primer
cctactcccagggctttgatt

SEQ ID NO: 27 - DNA, 20 bp, Artificial sequence
  Synthetic primer
ccgaggagatggagaatgag

SEQ ID NO: 28 - DNA, 20 bp, Artificial sequence
  Synthetic primer
ttcttccgcttccctttaca

(19) United States
(12) Patent Application Publication
(10) Pub. No.: US 2014/0377220 A1
(43) Pub. Date: Dec. 25, 2014

(54) GENE EXPRESSION SIGNATURES FOR STAGING AND PROGNOSIS OF PROSTATE, BREAST AND LEUKEMIA CANCERS

(71) Applicant: UTI LIMITED PARTNERSHIP, Calgary (CA)

(72) Inventor: Tarek A. Bismar, Calgary (CA)

(73) Assignee: UTI LIMITED PARTNERSHIP, Calgary, AB (CA)

(21) Appl. No.: 14/360,029

(22) PCT Filed: Nov. 21, 2012
(86) PCT No.: PCT/IB2012/003092

§ 371 Date: May 22, 2014

Related U.S. Application Data
(60) Provisional application No. 61/563,329, filed on Nov. 23, 2011.

(51) Int. Cl. C12Q 1/68; G01N 33/574

(52) U.S. Cl. CPC C12Q 1/6886; G01N 33/57496; G01N 33/57415; C12Q 2600/158
USPC 424/85.2; 435/7.1; 514/44 A; 435/6.11; 435/6.12

(57) ABSTRACT

The present invention is drawn to methods of assessing and treating cancers such as ERG-related, prostate, breast and leukemia, by examining the expression of combinations of particular genes disregulated in this disease state. The combinations of genes are selected from the following genes: inhibitor of growth family, member 3 (HSTG3); lymphoid enhancer-binding protein factor 1 (LEF1); frizzled-related protein (FRZB); annexin A4 (ANXA4); Meis homeobox 2 (MEIS2); syndecan binding protein (syntenin) (SDCBP); ankyrin 3, node of Ranvier (ankyrin G) (ANK3); chromodomain helicase DNA binding protein 5 (CHD5); phospholipase A2, group VII (platelet-activating factor acetylhydrolase, plasma) (PLA2G7); and wingless-type MMTV integration site family member 2 (WNT2).

This application claims benefit of priority to U.S. Provisional Application Ser. No. 61/563,329, filed Nov. 23, 2011, the entire contents of which are hereby incorporated by reference.

### BACKGROUND OF THE INVENTION

I. Field of the Invention

The present invention relates generally to the fields of biochemistry, molecular biology, and medicine. In certain aspects, the invention is related to to use of a panel of marker genes whose disregulated expression is diagnostic for stage of prostate, breast and leukemia cancers and patient prognosis.

II. Description of Related Art

The recent characterization of ERG gene rearrangements and its implication in molecular prostate cancer (PCA) subtyping has allowed us to initiate a road map to associate specific molecular subtypes with progression pathways with potential implication to targeted therapy similar to breast and other hematological malignancies (Tomlins et al., 2005).

Since the original report describing the translocation between the androgen regulated gene TMPRSS2 and one of the ETS family members of transcription factors (ERG, ETV1 and ETV4) (Tomlins et al., 2005), the list of novel recurrent gene rearrangements is increasing and now includes other members such as, ETV5, ELK4, SLC45A3 (solute carrier family 45, member 3), and NDRG1 (Tomlins et al., 2006; Helgeson et al., 2008; Esgueva et al., 2010; Rickman et al., 2009).

Several studies have also documented that PCA harboring ERG gene rearrangements are likely to harbor additional genetic aberrations, the most predominant of which are PTEN genomic deletions, reported to be present in ˜70% of such cases (Reid et al., 2010; Mosquera et al., 2007; Han et al., 2009a; Han et al, 2009b; Bismar et al., 2011). Furthermore, ERG rearrangements and PTEN genomic deletions seem to be implicted in disease progression and to reflect patients prognosis (Reid et al., 2010; Demichelis et al., 2007; Nam et al., 2007; Yoshimoto et al., 2007; Attard et al., 2008; Yoshimoto et al., 2008). In transgeneic mouse models, these two combined genetic alterations resulted in faster frank invasive carcinoma formation than in mice with ERG rearrangements or PTEN deletions alone (Carver et al., 2009; King et al., 2009; Squire, 2009).

Evidence is mounting that these recurrent gene rearrangements may also signify specific therapeutic responses, which could be the first step toward designing specific argeted molecular therapies in prostate cancer (Attard et al., 2009).

Recently, studies have been emerging in charecterizing novel genes associatited with ERG gene rearrangments that point toward potential pathways associated with ERG recurrent gene fusions. A novel mechanism has been put forth implicating androgen signaling involvment in the translocation of TMPRSS2 and ERG genomic loci, via promotion of the co-recruitment of androgen receptor and topoisomerase II beta (TOP2B) to sites of TMPRSS2-ERG genomic breakpoints (Haffner et al., 2010). Other reports have focused on specific downstream effects of ERG rearrangement and have identified several genes and biological processes that are deregulated as a result of abberant ERG activity, including: regulation of C-MYC expression (Hawksworth et al., 2010); EZH2 (Polycomb group protein) and Nkx3.1 (tumor suppressor gene) expression (Kunderfranco et al., 2010); enhanced metastasis of tumor cells through CXCR4 (cai et al., 2010); PLA2G7 (Vainio et al., 2011); NF-κB mediated transcription (Wang et al., 2010); TFF3 regulation (Rickman et al., 2010); epithelial-to-mesenchymal transition (EMT) (Leshem et al., 2011); NF-κB (Wang et al., 2011); and up-regulation of several WNT pathway members including frizzled-4 (FZD4) (Gupta et al., 2010). Other studis chose a differential expression approach betwen two groups of ERG rearranged and non rearranged tumors to identify down stream deregulated genes and genes with bilogical significance in patinets'prognosis (Barwick et al., 2010; Ribeiro et al., 2011).

Taken together, these studies and several others have demonstrated that a thorough interrogation and characterization of the molecular mechanisms that result in, or result from, ERG gene rearrangements would enable the identification of novel PCA biomarkers as well as the ability to predict disease progression, inform treatment options and allow for a better prediction of a given patient's prognosis.

### SUMMARY OF THE INVENTION

Thus, in accordance with the present invention, there is provided a method of predicting prognosis in a human subject diagnosed with prostate, leukemia or breast cancer comprising obtaining expression information for four or more of the following genes in a cancer sample obtained from said subject:
ING3, LEF1, FRZB, ANXA4, MEIS2,
SDCBP, ANK3, CHD5, PLA2G7, and WNT2,
   wherein a decrease in expression of ING3, LEF1, FRZB, MEIS2, and ANXA4 as compared to expression observed in non-cancer cells, and an increase in expression of SDCBP, ANK3, CHD5, PLA2G7 and WNT2, as compared to expression observed in non-cancer cells or control house-keeping genes, indicates a poor prognosis. The decrease and/or increase of expression may be least 0.5-fold. The method may further comprise staging said prostate, leukemia or breast cancer based on said expression information. The method may also further comprising obtaining information on PTEN expression or HER2 status.
   Obtaining expression information may comprise assessing protein expression, such as ELISA, RIA, immunohistochemistry, or mass spectrometry. Obtaining expression information expression may comprise assessing mRNA expression or gene methylation status, such as with quantitative RT-PCR, gene chip array expression, and/or Northern blotting. The expression observed in said non-cancer cell may be a pre-determined standard, or may be determined by assessing expression in a non-cancer cell from said subject. The method may comprise obtaining said sample. The cancer may be metastatic, localized or multi-drug resistant. Prognosis may be length of survival, such as disease-specific length of survival or overall survival. Prognosis may also be length of time to recurrence. The method may further comprising obtaining the cancer sample, such as by taking a biopsy or blood sample from said subject.

The method may further comprise making a treatment decision for said subject, and treating said subject, such as to give chemotherapy to a subject having a poor prognosis as compared to median or to not give chemotherapy to a subject having a favorable prognosis as compared to median. The method may also comprise treating said subject with adjuvant chemotherapy other therapy or offering watchful waiting protocol for good prognosis patients. Information on at least 5, 6, 7, 8, 9 or all 10 markers may be obtained.

In another embodiment, there is provided a method of predicting prognosis in a human subject diagnosed with breast cancer comprising obtaining expression information for the following genes in a cancer sample obtained from said subject:
ING3, LEF1, FRZB, and ANXA4,
   wherein a decrease in expression of ING3, LEF1, FRZB, and ANXA4 as compared to expression observed in non-cancer cells or control house-keeping genes, indicates a poor prognosis. The decrease and/or increase of expression may be least 0.5-fold. The method may further comprise staging said breast cancer based on said expression information. The method may also further comprising obtaining information on PTEN expression or HER2 status.

Obtaining expression information may comprise assessing protein expression, such as ELISA, RIA, immunohistochemistry, or mass spectrometry. Obtaining expression information expression may comprise assessing mRNA expression or gene methylation status, such as with quantitative RT-PCR, gene chip array expression, and/or Northern blotting. The expression observed in said non-cancer cell may be a pre-determined standard, or may be determined by assessing expression in a non-cancer cell from said subject. The method may comprise obtaining said sample. The cancer may be metastatic, localized or multi-drug resistant. Prognosis may be length of survival, such as disease-specific length of survival or overall survival. Prognosis may also be length of time to recurrence. The method may further comprising obtaining the cancer sample, such as by taking a biopsy or blood sample from said subject.

The method may further comprise making a treatment decision for said subject, and treating said subject, such as to give chemotherapy to a subject having a poor prognosis as compared to median or to not give chemotherapy to a subject having a favorable prognosis as compared to median. The method may also comprise treating said subject with adjuvant chemotherapy.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

The following figures form part of the present specification and are included to further demonstrate certain aspects of the present invention. The invention may be better understood by reference to one or more of these figures in combination with the detailed description of specific embodiments presented herein.

FIGS. 1A-B. Heat map of the ERG-gene signature across different samples. (FIG. 1A) Heatmap of the initially identified 28-gene signature across ERG1 and ERG0 samples in the present cohort. The 28 genes showed to group samples into three clusters. (FIG. 1B). Heatmap of the 10-gene signature across ERG1 and ERG0 samples in the Swedish cohort.

FIGS. 2A-C. Association of markers with ERG. (FIG. 2A) Heat map. (FIG. 2B) IHC plots. (FIG. 2C) Quantitative RT-PCR analysis of total RNA extracted from fresh-frozen localized prostate cancer samples previously characterized for ERG expression by qPCR and sorted into ERG negative and ERG positive cases. Mean relative expression of markers in ERG− and ERG+ groups is shown (n=3)+/−SEM. * indicates significant difference where p<0.05.

FIGS. 3A-B. Association of markers with cancer progression. (FIG. 3A) Selected images of tissue microarray elements representing immunohistochemical analysis of protein levels for the ten marker genes in benign prostate gland (BEN), localized prostate cancer (PCA) and castration-resistant PCA (CRPCA). Relative levels of proteins as assessed by blinded pathology analysis of tissue microarrays (n=86 patients) are provided to the right. (FIG. 3B) Quantitative RT-PCR analysis of total RNA extracted from paired benign prostate (BEN) and localized prostate cancer (PCA, n=10), and lymph node metastases (LN Mets, n=3). Mean relative expression+/−SEM of markers in each group is shown. * indicates significant difference from benign samples; ** indicates significant difference from both benign and localized PCA samples where p<0.05.

FIGS. 4A-C. Descriminative ability of the 10-gene model and its association to cancer progression. (FIG. 4A) Hierarcical clustering of TMA samples showed that BN samples are cleary distinct from CA and HRPCA that have similar expression profile. (FIG. 4B) ROC curve analysis for BN vs CA and HRPCA classification. (FIG. 4C) Prinicipal component analysis reveals that BN samples are distinct from CA and HRPCA samples.

FIGS. 5A-B. Kaplan-Meier survival curves for patients in Swedish cohort. (FIG. 5A) ERG status showed to be significant to separate patients into two groups. (FIG. 5B) The 10-gene model showed significant association with the survival outcome, ERG1-like patients are the cluster of patients enriched with ERG1 samples.

FIGS. 6A-C. Kaplan-Meier survival curves for patients in Glinsky data. (FIG. 6A) Patients are grouped based on ERG status. (FIG. 6B) Patients are grouped into high and low risk based on (Varambally et al., 2011). (FIG. 6C) Samples are grouped based on the 10-gene model.

FIGS. 7A-B. Kaplan-Meier survival curves for recurrence time in Tylor. (FIG. 7A) Patients are grouped based on ERG expression. (FIG. 7B) Patients are grouped based on 10-gene model.

FIGS. 8A-B. Kaplan-Meier survival curves for survival in leukemia data. (FIG. 8A) Patients are grouped based on ERG expression. (FIG. 8B) Patients are grouped based on 10-gene model.

FIGS. 9A-D. Kaplan-Meier survival curves for survival in breast data. (FIG. 9A) Patients are grouped based on ERG expression. (FIG. 9B) Patients are grouped based on 10-gene model. (FIGS. 9C-D) Survival plot generated from GOBO tool analyzing the effect of ERG expression and ERG like signature on survival in large breast cancer cohorts.

### DESCRIPTION OF ILLUSTRATIVE EMBODIMENTS

In the current study, the inventor used a differential expression microarray approach combined with a bioinformatic interrogation of ERG-negative and ERG-positive tumors using the singular value decomposition (SVD) method to identify novel genes associated with disease progression and with prognostic implication for cancer recurrence and disease specific mortality. The inventor further characterized potential pathways implicated in prostate cancer progression relevant to ERG gene rearrangements and identify multi-gene model with prognostic significance across several tumor types which suggest implication in disease progression pathways in various malignancies.

ERG gene rearrangements are one of the most common gene alterations affecting prostate cancer. Expression profile studies point out that ERG tumors represent a subset of prostate tumors tat share specific progression pathways and could be of prognostic and potential theraputic significance. Data from transgeneic mice confirm that ERG to be synergetic to other genetic alteration in prostate cancer in developing frank tumors. As prostate cancer is one of the most hetergenous tumors, it is expected that predicting tumor prgression would be more acheivable and reliable based on multi gene model rather than individual gens. This is consisitent with what has been found in breast cancer with the 21-gen model currently being implemented clinically to stratify patients into different risk groups to implment chemotherapy.

In this study, the inventor has identified and characterized a panel of 10 genes that show potential for further development as a signature for aggressive and indolent prostate cancer. This panel was identified by a combination of SVD bioinformatic analysis and several network-based search methods and validated on several well annotated and large cohorts. The signature identified was not only applicable to prostate cancer, but rather showed prognostic significance in other tumors which shows differecne in ERG gene expression. The signature identifed was confirmed to be more accurate than ERG gene expression alone which at times showed to be unreliable (based on the Swedish breast cancer cohort). This 10-gene signature could be the initial step in improving the ablitity to distinguish aggressive and indolent prostate cancer prior to implmenting definite therapy. This would also enable one to offer selected patients a tailored therapy based on the signature of their tumors at the time of prostate biopsy, coupled with clinical and pathological information, thereby avoiding overtreatment of those patients and potential harmful side effects.

In summary, the inventor has identified and validated an ERG-like signature of ERG-related cancer that is more robust than ERG expression alone. This signature, coupled with clinical and pathological findings in prostate biopsy, can enable one to separate aggressive from indolent disease and to identify patients at highest risk of cancer mortality in prostate cancer and poetntially breast cancer and leukemia as well. These and other aspects of the invention are described in detail below.

### I. ERG-Involved Cancers

A. Prostate Cancer

Prostate cancer is a form of cancer that develops in the prostate, a gland in the male reproductive system. Most prostate cancers are slow growing; however, there are cases of aggressive prostate cancers. The cancer cells may metastasize (spread) from the prostate to other parts of the body, particularly the bones and lymph nodes. Prostate cancer may cause pain, difficulty in urinating, problems during sexual intercourse, or erectile dysfunction. Other symptoms can potentially develop during later stages of the disease.

Rates of detection of prostate cancers vary widely across the world, with South and East Asia detecting less frequently than in Europe, and especially the United States. Prostate cancer tends to develop in men over the age of fifty and although it is one of the most prevalent types of cancer in men, many never have symptoms, undergo no therapy, and eventually die of other causes. This is because cancer of the prostate is, in most cases, slow-growing, symptom-free, and since men with the condition are older they often die of causes unrelated to the prostate cancer, such as heart/circulatory disease, pneumonia, other unconnected cancers, or old age. About two-thirds of cases are slow growing, the other third more aggressive and fast developing.

Many factors, including genetics and diet, have been implicated in the development of prostate cancer. The presence of prostate cancer may be indicated by symptoms, physical examination, prostate-specific antigen (PSA), or biopsy. The PSA test increases cancer detection but does not decrease mortality. Moreover, prostate test screening is controversial at the moment and may lead to unnecessary, even harmful, consequences in some patients. Nonetheless, suspected prostate cancer is typically confirmed by taking a biopsy of the prostate and examining it under a microscope. Further tests, such as CT scans and bone scans, may be performed to determine whether prostate cancer has spread.

Treatment options for prostate cancer with intent to cure are primarily surgery, radiation therapy, stereotactic radiosurgery, and proton therapy. Other treatments, such as hormonal therapy, chemotherapy, cryosurgery, and high intensity focused ultrasound (HIFU) also exist, although not FDA approved, depending on the clinical scenario and desired outcome.

The age and underlying health of the man, the extent of metastasis, appearance under the microscope, and response of the cancer to initial treatment are important in determining the outcome of the disease. The decision whether or not to treat localized prostate cancer (a tumor that is contained within the prostate) with curative intent is a patient trade-off between the expected beneficial and harmful effects in terms of patient survival and quality of life.

The prostate is a part of the male reproductive system that helps make and store seminal fluid. In adult men, a typical prostate is about three centimeters long and weighs about twenty grams. It is located in the pelvis, under the urinary bladder and in front of the rectum. The prostate surrounds part of the urethra, the tube that carries urine from the bladder during urination and semen during ejaculation. Because of its location, prostate diseases often affect urination, ejaculation, and rarely defecation. The prostate contains many small glands which make about twenty percent of the fluid constituting semen. In prostate cancer, the cells of these prostate glands mutate into cancer cells. The prostate glands require male hormones, known as androgens, to work properly. Androgens include testosterone, which is made in the testes; dehydroepiandrosterone, made in the adrenal glands; and dihydrotestosterone, which is converted from testosterone within the prostate itself. Androgens are also responsible for secondary sex characteristics such as facial hair and increased muscle mass.

An important part of evaluating prostate cancer is determining the stage, or how far the cancer has spread. Knowing the stage helps define prognosis and is useful when selecting therapies. The most common system is the four-stage TNM system (abbreviated from Tumor/Nodes/Metastases). Its components include the size of the tumor, the number of involved lymph nodes, and the presence of any other metastases.

The most important distinction made by any staging system is whether or not the cancer is still confined to the prostate. In the TNM system, clinical T1 and T2 cancers are found only in the prostate, while T3 and T4 cancers have spread elsewhere. Several tests can be used to look for evidence of spread. These include computed tomography to evaluate spread within the pelvis, bone scans to look for spread to the bones, and endorectal coil magnetic resonance imaging to closely evaluate the prostatic capsule and the seminal vesicles. Bone scans should reveal osteoblastic appearance due to increased bone density in the areas of bone metastasis—opposite to what is found in many other cancers that metastasize.

After a prostate biopsy, a pathologist looks at the samples under a microscope. If cancer is present, the pathologist reports the grade of the tumor. The grade tells how much the tumor tissue differs from normal prostate tissue and suggests how fast the tumor is likely to grow. The Gleason system is used to grade prostate tumors from 2 to 10, where a Gleason score of 10 indicates the most abnormalities. The pathologist assigns a number from 1 to 5 for the most common pattern observed under the microscope, then does the same for the second-most-common pattern. The sum of these two numbers is the Gleason score. The Whitmore-Jewett stage is another method sometimes used.

Early prostate cancer usually causes no symptoms. Often it is diagnosed during the workup for an elevated PSA noticed during a routine checkup. Sometimes, however, prostate cancer does cause symptoms, often similar to those of diseases such as benign prostatic hyperplasia. These include frequent urination, nocturia (increased urination at night), difficulty starting and maintaining a steady stream of urine, hematuria (blood in the urine), and dysuria (painful urination).

Prostate cancer is associated with urinary dysfunction as the prostate gland surrounds the prostatic urethra. Changes within the gland, therefore, directly affect urinary function. Because the vas deferens deposits seminal fluid into the prostatic urethra, and secretions from the prostate gland itself are included in semen content, prostate cancer may also cause problems with sexual function and performance, such as difficulty achieving erection or painful ejaculation.

Advanced prostate cancer can spread to other parts of the body, possibly causing additional symptoms. The most common symptom is bone pain, often in the vertebrae (bones of the spine), pelvis, or ribs. Spread of cancer into other bones such as the femur is usually to the proximal part of the bone. Prostate cancer in the spine can also compress the spinal cord, causing leg weakness and urinary and fecal incontinence.

Ultrasound (US) and Magnetic Resonance Imaging (MRI) are the two main imaging methods used for prostate cancer detection. Urologists use transrectal ultrasound during prostate biopsy and can sometimes see a hypoechoic area. But US has poor tissue resolution and thus, is generally not clinically used. In contrast, prostate MRI has superior soft tissue resolution. MRI is a type of imaging that uses magnetic fields to locate and characterize prostate cancer. Multi-parametric prostate MRI consists of four types of MRI sequences called T2 weighted imaging, T1 weighted imaging, Diffusion Weighted Imaging, MR Spectrocopic Imaging and Dynamic-Contrast Enhanced Imaging. Genitourinary radiologists use multi-parametric MRI to locate and identify prostate cancer. Currently, MRI is used to identify targets for prostate biopsy using fusion MRI with ultrasound (US) or MRI-guidance alone. In men who are candidates for active surveillance, fusion MR/US guided prostate biopsy detected 33% of cancers compared to 7% with standard ultrasound guided biopsy. Prostate MRI is also used for surgical planning for men undergoing robotic prostatectomy. It has also shown to help surgeons decide whether to resect or spare the neurovascular bundle, determine return to urinary continence and help assess surgical difficulty. Some prostate advocacy groups believe prostate MRI should be used to screen for prostate cancer.

If cancer is suspected, a biopsy is offered expediently. During a biopsy a urologist or radiologist obtains tissue samples from the prostate via the rectum. A biopsy gun inserts and removes special hollow-core needles (usually three to six on each side of the prostate) in less than a second. Prostate biopsies are routinely done on an outpatient basis and rarely require hospitalization. Fifty-five percent of men report discomfort during prostate biopsy.

The tissue samples are then examined under a microscope to determine whether cancer cells are present, and to evaluate the microscopic features (or Gleason score) of any cancer found. Prostate specific membrane antigen is a transmembrane carboxypeptidase and exhibits folate hydrolase activity. This protein is overexpressed in prostate cancer tissues and is associated with a higher Gleason score.

Tissue samples can be stained for the presence of PSA and other tumor markers in order to determine the origin of malignant cells that have metastasized. Small cell carcinoma is a very rare (1%) type of prostate cancer that cannot be diagnosed using the PSA. As of 2009 researchers are trying to determine the best way to screen for this type of prostate cancer because it is a relatively unknown and rare type of prostate cancer but very serious and quick to spread to other parts of the body. Possible methods include chromatographic separation methods by mass spectrometry, or protein capturing by immunoassays or immunized antibodies. The test method will involve quantifying the amount of the biomarker PCI, with reference to the Gleason Score. Not only is this test quick, it is also sensitive. It can detect patients in the diagnostic grey zone, particularly those with a serum free to total Prostate Specific Antigen ratio of 10-20%.

The oncoprotein BCL-2, has been associated with the development of androgen-independent prostate cancer due to its high levels of expression in androgen-independent tumours in advanced stages of the pathology. The upregulation of BCL-2 after androgen ablation in prostate carcinoma cell lines and in a castrated-male rat model further established a connection between BCL-2 expression and prostate cancer progression. The expression of Ki-67 by immunohistochemistry may be a significant predictor of patient outcome for men with prostate cancer. ERK5 is a protein that may be used as a marker. ERK5 is present in abnormally high levels of prostate cancer, including invasive cancer which has spread to other parts of the body. It is also present in relapsed cancer following previous hormone therapy. Research shows that reducing the amount of ERK5 found in cancerous cells reduces their invasiveness.

Treatment for prostate cancer may involve active surveillance (monitoring for tumor progress or symptoms), surgery (i.e., radical prostatectomy), radiation therapy including brachytherapy (prostate brachytherapy) and external beam radiation therapy, High-intensity focused ultrasound (HIFU), chemotherapy, oral chemotherapeutic drugs (Temozolomide/TMZ), cryosurgery, hormonal therapy, or some combination. Which option is best depends on the stage of the disease, the Gleason score, and the PSA level. Other important factors are age, general health, and patient views about potential treatments and their possible side-effects. Because all treatments can have significant side-effects, such as erectile dysfunction and urinary incontinence, treatment discussions often focus on balancing the goals of therapy with the risks of lifestyle alterations. Prostate cancer patients are strongly recommended to work closely with their physicians and use a combination of the treatment options when managing their prostate cancer.

Because of PSA screening, almost 90% of patients are diagnosed when the cancer is localized to the prostate gland and its removal by surgery or radiotherapy will in most cases lead to a cure. Because of this almost 94% of U.S. patients choose treatment. However, in 50% to 75% of these patients the cancer would not have affected their survival even without treatment, and by accepting treatment they have a high chance of sexual, urinary, and bowel side effects. For instance, two-thirds of treated patients cannot get sufficient erections for intercourse, and almost a third have urinary leakage. However, some cancers will grow faster and prostate cancer is the second most common reason of cancer death in U.S. men, after lung cancer. Even the most intelligent and educated patient faces this uncertainty, and 1 in 6 men will be diagnosed with prostate cancer in their life time.

The selection of treatment options may be a complex decision involving many factors. For example, radical prostatectomy after primary radiation failure is a very technically challenging surgery and may not be an option, while salvage radiation therapy after surgical failure may have many complications. This may enter into the treatment decision. If the cancer has spread beyond the prostate, treatment options significantly change, so most doctors that treat prostate cancer use a variety of nomograms to predict the probability of spread. Treatment by watchful waiting/active surveillance, external beam radiation therapy, brachytherapy, cryosurgery, HIFU, and surgery are, in general, offered to men whose cancer remains within the prostate. Hormonal therapy and chemotherapy are often reserved for disease that has spread beyond the prostate. However, there are exceptions: radiation therapy may be used for some advanced tumors, and hormonal therapy is used for some early stage tumors. Cryotherapy (the process of freezing the tumor), hormonal therapy, and chemotherapy may also be offered if initial treatment fails and the cancer progresses.

Most hormone dependent cancers become refractory after one to three years and resume growth despite hormone therapy. Previously considered “hormone-refractory prostate cancer” or “androgen-independent prostate cancer,” the term castration-resistant has replaced “hormone refractory” because while they are no longer responsive to castration treatment (reduction of available androgen/testosterone/DHT by chemical or surgical means), these cancers still show reliance upon hormones for androgen receptor activation. Before 2004, all treatments for castration-resistant prostate cancer (CRPC) were considered palliative and not shown to prolong survival. However, there are now several treatments available to treat CRPC that improve survival.

The cancer chemotherapic docetaxel has been used as treatment for (CRPC) with a median survival benefit of 2 to 3 months. Docetaxel's FDA approval in 2004 was significant as it was the first treatment proven to prolong survival in CRPC. In 2010, the FDA approved a second-line chemotherapy treatment known as cabazitaxel. Off-label use of the oral drug ketoconazole is sometimes used as a way to further manipulate hormones with a therapeutic effect in CRPC. However, many side effects are possible with this drug and abiraterone is likely to supplant usage since it has a similar mechanism of action with less toxic side effects. A combination of bevacizumab (Avastin), docetaxel, thalidomide and prednisone appears effective in the treatment of CRPC. The immunotherapy treatment with sipuleucel-T is also effective in the treatment of CRPC with a median survival benefit of 4.1 months.

In patients who undergo treatment, the most important clinical prognostic indicators of disease outcome are stage, pre-therapy PSA level, and Gleason score. In general, the higher the grade and the stage, the poorer the prognosis. Nomograms can be used to calculate the estimated risk of the individual patient. The predictions are based on the experience of large groups of patients suffering from cancers at various stages.

In 1941, Charles Huggins reported that androgen ablation therapy causes regression of primary and metastatic androgen-dependent prostate cancer. Androgen ablation therapy causes remission in 80-90% of patients undergoing therapy, resulting in a median progression-free survival of 12 to 33 months. After remission, an androgen-independent phenotype typically emerges, wherein the median overall survival is 23-37 months from the time of initiation of androgen ablation therapy. The actual mechanism contributes to the progression of prostate cancer is not clear and may vary between individual patient. A few possible mechanisms have been proposed.

Many prostate cancers are not destined to be lethal, and most men will ultimately die from causes other than of the disease. Decisions about treatment type and timing may, therefore, be informed by an estimation of the risk that the tumor will ultimately recur after treatment and/or progress to metastases and mortality. Several tools are available to help predict outcomes, such as pathologic stage and recurrence after surgery or radiation therapy. Most combine stage, grade, and PSA level, and some also add the number or percent of biopsy cores positive, age, and/or other information.

The D'Amico classification stratifies men by low, intermediate, or high risk based on stage, grade, and PSA. It is used widely in clinical practice and research settings. The major downside to the 3-level system is that it does not account for multiple adverse parameters (e.g., high Gleason score and high PSA) in stratifying patients.

The Partin tables predict pathologic outcomes (margin status, extraprostatic extension, and seminal vesicle invasion) based on the same three variables and are published as lookup tables.

The Kattan nomograms predict recurrence after surgery and/or radiation therapy, based on data available either at time of diagnosis or after surgery. The nomograms can be calculated using paper graphs or software available on a website or for handheld computers. The Kattan score represents the likelihood of remaining free of disease at a given time interval following treatment.

The UCSF Cancer of the Prostate Risk Assessment (CAPRA) score predicts both pathologic status and recurrence after surgery. It offers comparable accuracy as the Kattan preoperative nomogram, and can be calculated without paper tables or a calculator. Points are assigned based on PSA, Grade, stage, age, and percent of cores positive; the sum yields a 0-10 score, with every 2 points representing roughly a doubling of risk of recurrence. The CAPRA score was derived from community-based data in the CaPSURE database. It has been validated among over 10,000 prostatectomy patients, including patients from CaPSURE; the SEARCH registry, representing data from several Veterans Administration and active military medical centers; a multi-institutional cohort in Germany; and the prostatectomy cohort at Johns Hopkins University. More recently, it has been shown to predict metastasis and mortality following prostatectomy, radiation therapy, watchful waiting, or androgen deprivation therapy.

B. Breast Cancer

Breast cancer (malignant breast neoplasm) is cancer originating from breast tissue, most commonly from the inner lining of milk ducts or the lobules that supply the ducts with milk. Cancers originating from ducts are known as ductal carcinomas; those originating from lobules are known as lobular carcinomas. Breast cancer is a disease of humans and other mammals; while the overwhelming majority of cases in humans are women, men can also develop breast cancer. Worldwide, breast cancer comprises 22.9% of all cancers (excluding non-melanoma skin cancers) in women. In 2008, breast cancer caused 458,503 deaths worldwide (13.7% of cancer deaths in women). Breast cancer is more than 100 times more common in women than breast cancer in men, although males tend to have poorer outcomes due to delays in diagnosis.

The size, stage, rate of growth, and other characteristics of the tumor determine the kinds of treatment. Treatment may include surgery, drugs (hormonal therapy and chemotherapy), radiation and/or immunotherapy. Surgical removal of the tumor provides the single largest benefit, with surgery alone being capable of producing a cure in many cases. To somewhat increase the likelihood of long-term disease-free survival, several chemotherapy regimens are commonly given in addition to surgery. Most forms of chemotherapy kill cells that are dividing rapidly anywhere in the body, and as a result cause temporary hair loss and digestive disturbances. Radiation is indicated especially after breast conserving surgery and substantially improves local relapse rates and in many circumstances also overall survival. Some breast cancers are sensitive to hormones such as estrogen and/or progesterone, which makes it possible to treat them by blocking the effects of these hormones. Prognosis and survival rate varies greatly depending on cancer type, staging and treatment, 5-year relative survival varies from 98% to 23%, with an overall survival rate of 85%.

The first noticeable symptom of breast cancer is typically a lump that feels different from the rest of the breast tissue. More than 80% of breast cancer cases are discovered when the woman feels a lump. The earliest breast cancers are detected by a mammogram. Lumps found in lymph nodes located in the armpits can also indicate breast cancer. Indications of breast cancer other than a lump may include changes in breast size or shape, skin dimpling, nipple inversion, or spontaneous single-nipple discharge. Pain (“mastodynia”) is an unreliable tool in determining the presence or absence of breast cancer, but may be indicative of other breast health issues.

Inflammatory breast cancer is a particular type of breast cancer which can pose a substantial diagnostic challenge. Symptoms may resemble a breast inflammation and may include itching, pain, swelling, nipple inversion, warmth and redness throughout the breast, as well as an orange-peel texture to the skin referred to as peau d'orange; the absence of a discernible lump delays detection dangerously.

Another reported symptom complex of breast cancer is Paget's disease of the breast. This syndrome presents as eczematoid skin changes such as redness and mild flaking of the nipple skin. As Paget's advances, symptoms may include tingling, itching, increased sensitivity, burning, and pain. There may also be discharge from the nipple. Approximately half of women diagnosed with Paget's also have a lump in the breast.

In rare cases, what initially appears as a fibroadenoma (hard movable lump) could in fact be a phyllodes tumor. Phyllodes tumors are formed within the stroma (connective tissue) of the breast and contain glandular as well as stromal tissue. Phyllodes tumors are not staged in the usual sense; they are classified on the basis of their appearance under the microscope as benign, borderline, or malignant.

Occasionally, breast cancer presents as metastatic disease, that is, cancer that has spread beyond the original organ. Metastatic breast cancer will cause symptoms that depend on the location of metastasis. Common sites of metastasis include bone, liver, lung and brain. Unexplained weight loss can occasionally herald an occult breast cancer, as can symptoms of fevers or chills. Bone or joint pains can sometimes be manifestations of metastatic breast cancer, as can jaundice or neurological symptoms. These symptoms are called non-specific, meaning they could be manifestations of many other illnesses.

Most symptoms of breast disorders, including most lumps, do not turn out to represent underlying breast cancer. Benign breast diseases such as mastitis and fibroadenoma of the breast are more common causes of breast disorder symptoms. Nevertheless, the appearance of a new symptom should be taken seriously by both patients and their doctors, because of the possibility of an underlying breast cancer at almost any age.

Breast cancer, like other cancers, occurs because of an interaction between the environment and a defective gene. Normal cells divide as many times as needed and stop. They attach to other cells and stay in place in tissues. Cells become cancerous when mutations destroy their ability to stop dividing, to attach to other cells and to stay where they belong. When cells divide, their DNA is normally copied with many mistakes. Error-correcting proteins fix those mistakes. The mutations known to cause cancer, such as p53, BRCA1 and BRCA2, occur in the error-correcting mechanisms. These mutations are either inherited or acquired after birth. Presumably, they allow the other mutations, which allow uncontrolled division, lack of attachment, and metastasis to distant organs. Normal cells will commit cell suicide (apoptosis) when they are no longer needed. Until then, they are protected from cell suicide by several protein clusters and pathways. One of the protective pathways is the PI3K/AKT pathway; another is the RAS/MEK/ERK pathway. Sometimes the genes along these protective pathways are mutated in a way that turns them permanently “on,” rendering the cell incapable of committing suicide when it is no longer needed. This is one of the steps that causes cancer in combination with other mutations. Normally, the PTEN protein turns off the PI3K/AKT pathway when the cell is ready for cell suicide. In some breast cancers, the gene for the PTEN protein is mutated, so the PI3K/AKT pathway is stuck in the “on” position, and the cancer cell does not commit suicide. Mutations that can lead to breast cancer have been experimentally linked to estrogen exposure. Abnormal growth factor signaling in the interaction between stromal cells and epithelial cells can facilitate malignant cell growth. In breast adipose tissue, overexpression of leptin leads to increased cell proliferation and cancer.

In the United States, 10 to 20 percent of patients with breast cancer and patients with ovarian cancer have a first- or second-degree relative with one of these diseases. Mutations in either of two major susceptibility genes, breast cancer susceptibility gene 1 (BRCA1) and breast cancer susceptibility gene 2 (BRCA2), confer a lifetime risk of breast cancer of between 60 and 85 percent and a lifetime risk of ovarian cancer of between 15 and 40 percent. However, mutations in these genes account for only 2 to 3 percent of all breast cancers.

Both mammography and clinical breast exam, also used for screening, can indicate an approximate likelihood that a lump is cancer, and may also detect some other lesions. When the tests are inconclusive Fine Needle Aspiration and Cytology (FNAC) may be used. FNAC may be done in a GP's office using local anaesthetic if required, involves attempting to extract a small portion of fluid from the lump. Clear fluid makes the lump highly unlikely to be cancerous, but bloody fluid may be sent off for inspection under a microscope for cancerous cells. Together, these three tools can be used to diagnose breast cancer with a good degree of accuracy. Other options for biopsy include core biopsy, where a section of the breast lump is removed, and an excisional biopsy, where the entire lump is removed. In addition vacuum-assisted breast biopsy (VAB) may help diagnose breast cancer among patients with a mammographically detected breast in women.

Breast cancers are classified by several grading systems. Each of these influences the prognosis and can affect treatment response. Description of a breast cancer optimally includes all of these factors.
Histopathology. Breast cancer is usually classified primarily by its histological appearance. Most breast cancers are derived from the epithelium lining the ducts or lobules, and these cancers are classified as ductal or lobular carcinoma. Carcinoma in situ is growth of low grade cancerous or precancerous cells within a particular tissue compartment such as the mammary duct without invasion of the surrounding tissue. In contrast, invasive carcinoma does not confine itself to the initial tissue compartment.
Grade. Grading compares the appearance of the breast cancer cells to the appearance of normal breast tissue. Normal cells in an organ like the breast become differentiated, meaning that they take on specific shapes and forms that reflect their function as part of that organ. Cancerous cells lose that differentiation. In cancer, the cells that would normally line up in an orderly way to make up the milk ducts become disorganized. Cell division becomes uncontrolled. Cell nuclei become less uniform. Pathologists describe cells as well differentiated (low grade), moderately differentiated (intermediate grade), and poorly differentiated (high grade) as the cells progressively lose the features seen in normal breast cells. Poorly differentiated cancers have a worse prognosis.
Stage. Breast cancer staging using the TNM system is based on the size of the tumor (T), whether or not the tumor has spread to the lymph nodes (N) in the armpits, and whether the tumor has metastasized (M) (i.e. spread to a more distant part of the body). Larger size, nodal spread, and metastasis have a larger stage number and a worse prognosis. The main stages are:
Stage 0 is a pre-cancerous or marker condition, either ductal carcinoma in situ (DCIS) or lobular carcinoma in situ (LCIS).
Stages 1-3 are within the breast or regional lymph nodes.
Stage 4 is ‘metastatic’ cancer that has a less favorable prognosis.
Receptor status. Breast cancer cells have receptors on their surface and in their cytoplasm and nucleus. Chemical messengers such as hormones bind to receptors, and this causes changes in the cell. Breast cancer cells may or may not have three important receptors: estrogen receptor (ER), progesterone receptor (PR), and HER2/neu. ER+ cancer cells depend on estrogen for their growth, so they can be treated with drugs to block estrogen effects (e.g., tamoxifen), and generally have a better prognosis. HER2+ breast cancer had a worse prognosis, but HER2+ cancer cells respond to drugs such as the monoclonal antibody trastuzumab (in combination with conventional chemotherapy), and this has improved the prognosis significantly. Cells with none of these receptors are called basal-like or triple negative.
DNA assays. DNA testing of various types including DNA microarrays have compared normal cells to breast cancer cells. The specific changes in a particular breast cancer can be used to classify the cancer in several ways, and may assist in choosing the most effective treatment for that DNA type.
   Breast cancer is usually treated with surgery and then possibly with chemotherapy or radiation, or both. A multidisciplinary approach is preferable. Hormone positive cancers are treated with long term hormone blocking therapy. Treatments are given with increasing aggressiveness according to the prognosis and risk of recurrence. Stage 1 cancers (and DCIS) have an excellent prognosis and are generally treated with lumpectomy and sometimes radiation. HER2+ cancers should be treated with the trastuzumab (Herceptin) regime. Chemotherapy is uncommon for other types of stage 1 cancers. Stage 2 and 3 cancers with a progressively poorer prognosis and greater risk of recurrence are generally treated with surgery (lumpectomy or mastectomy with or without lymph node removal), chemotherapy (plus trastuzumab for HER2+ cancers) and sometimes radiation (particularly following large cancers, multiple positive nodes or lumpectomy). Stage 4, metastatic cancer (i.e., spread to distant sites), has poor prognosis and is managed by various combination of all treatments from surgery, radiation, chemotherapy and targeted therapies. 10 year survival rate is 5% without treatment and 10% with optimal treatment.

Surgery involves the physical removal of the tumor, typically along with some of the surrounding tissue and frequently sentinel node biopsy. Standard surgeries include mastectomy (removal of the whole breast), quadrantectomy (removal of one quarter of the breast), lumpectomy (removal of a small part of the breast). If the patient desires, then breast reconstruction surgery, a type of cosmetic surgery, may be performed to create an aesthetic appearance. In other cases, women use breast prostheses to simulate a breast under clothing, or choose a flat chest.

Drugs used after and in addition to surgery are called adjuvant therapy. Not all of these are appropriate for every person with breast cancer. Chemotherapy or other types of therapy prior to surgery are called neoadjuvant therapy. There are currently three main groups of medications used for adjuvant breast cancer treatment: hormone blocking therapy, chemotherapy, and monoclonal antibodies.
Hormone blocking therapy. Some breast cancers require estrogen to continue growing. They can be identified by the presence of estrogen receptors (ER+) and progesterone receptors (PR+) on their surface (sometimes referred to together as hormone receptors). These ER+ cancers can be treated with drugs that either block the receptors, e.g., tamoxifen (Nolvadex), or alternatively block the production of estrogen with an aromatase inhibitor, e.g., anastrozole (Arimidex) or letrozole (Femara). Aromatase inhibitors, however, are only suitable for post-menopausal patients.
Chemotherapy. Chemotherapies are predominately used for stage 2-4 disease, being particularly beneficial in estrogen receptor-negative (ER−) disease. They are given in combinations, usually for 3-6 months. One of the most common treatments is cyclophosphamide plus doxorubicin (Adriamycin), known as AC. Most chemotherapy medications work by destroying fast-growing and/or fast-replicating cancer cells either by causing DNA damage upon replication or other mechanisms; these drugs also damage fast-growing normal cells where they cause serious side effects. Damage to the heart muscle is the most dangerous complication of doxorubicin. Sometimes a taxane drug, such as docetaxel, is added, and the regime is then known as CAT; taxane attacks the microtubules in cancer cells. Another common treatment, which produces equivalent results, is cyclophosphamide, methotrexate, and fluorouracil (CMF). (Chemotherapy can literally refer to any drug, but it is usually used to refer to traditional non-hormone treatments for cancer.)
Monoclonal antibodies. Monoclonal antibodies represent a relatively recent development in HER2+ breast cancer treatment. Approximately 15-20 percent of breast cancers have an amplification of the HER2/neu gene or overexpression of its protein product. This receptor is normally stimulated by a growth factor which causes the cell to divide; in the absence of the growth factor, the cell will normally stop growing. Overexpression of this receptor in breast cancer is associated with increased disease recurrence and worse prognosis. Trastuzumab (Herceptin®), a monoclonal antibody to HER2, has improved the 5 year disease free survival of stage 1-3 HER2+ breast cancers to about 87% (overall survival 95%). Trastuzumab, however, is expensive, and approx 2% of patients suffer significant heart damage; it is otherwise well tolerated, with far milder side effects than conventional chemotherapy. Other monoclonal antibodies are also undergoing clinical trials.
   A recent analysis indicated that aspirin may reduce mortality from breast cancer. Radiotherapy is given after surgery to the region of the tumor bed and regional lymph nodes, to destroy microscopic tumor cells that may have escaped surgery. It may also have a beneficial effect on tumor microenvironment. Radiation therapy can be delivered as external beam radiotherapy or as brachytherapy (internal radiotherapy). Conventionally radiotherapy is given after the operation for breast cancer. Radiation can also be given at the time of operation on the breast cancer—intraoperatively.

C. Leukemia

Leukemia is a type of cancer of the blood or bone marrow characterized by an abnormal increase of white blood cells. Leukemia is a broad term covering a spectrum of diseases. In turn, it is part of the even broader group of diseases affecting the blood, bone marrow and lymphoid system, which are all known as hematological neoplasms. In 2000, approximately 256,000 children and adults around the world developed some form of leukemia, and 209,000 died from it. About 90% of all leukemias are diagnosed in adults.

Clinically and pathologically, leukemia is subdivided into a variety of large groups. The first division is between its acute and chronic forms. Acute leukemia is characterized by a rapid increase in the numbers of immature blood cells. Crowding due to such cells makes the bone marrow unable to produce healthy blood cells. Immediate treatment is required in acute leukemia due to the rapid progression and accumulation of the malignant cells, which then spill over into the bloodstream and spread to other organs of the body. Acute forms of leukemia are the most common forms of leukemia in children. Chronic leukemia is characterized by the excessive build up of relatively mature, but still abnormal, white blood cells. Typically taking months or years to progress, the cells are produced at a much higher rate than normal cells, resulting in many abnormal white blood cells in the blood. Whereas acute leukemia must be treated immediately, chronic forms are sometimes monitored for some time before treatment to ensure maximum effectiveness of therapy. Chronic leukemia mostly occurs in older people, but can theoretically occur in any age group.

Additionally, the diseases are subdivided according to which kind of blood cell is affected. This split divides leukemias into lymphoblastic or lymphocytic leukemias and myeloid or myelogenous leukemias. In lymphoblastic or lymphocytic leukemias, the cancerous change takes place in a type of marrow cell that normally goes on to form lymphocytes, which are infection-fighting immune system cells. Most lymphocytic leukemias involve a specific subtype of lymphocyte, the B cell. In myeloid or myelogenous leukemias, the cancerous change takes place in a type of marrow cell that normally goes on to form red blood cells, some other types of white cells, and platelets. Combining these two classifications provides a total of four main categories. Within each of these four main categories, there are typically several subcategories. Finally, some rarer types are usually considered to be outside of this classification scheme.

Acute lymphoblastic leukemia (ALL) is the most common type of leukemia in young children. This disease also affects adults, especially those age 65 and older. Standard treatments involve chemotherapy and radiotherapy. The survival rates vary by age: 85% in children and 50% in adults. Subtypes include precursor B acute lymphoblastic leukemia, precursor T acute lymphoblastic leukemia, Burkitt's leukemia, and acute biphenotypic leukemia.

Chronic lymphocytic leukemia (CLL) most often affects adults over the age of 55. It sometimes occurs in younger adults, but it almost never affects children. Two-thirds of affected people are men. The five-year survival rate is 75%. It is incurable, but there are many effective treatments. One subtype is B-cell prolymphocytic leukemia, a more aggressive disease.

Acute myelogenous leukemia (AML) occurs more commonly in adults than in children, and more commonly in men than women. AML is treated with chemotherapy. The five-year survival rate is 40%. Subtypes of AML include acute promyelocytic leukemia, acute myeloblastic leukemia, and acute megakaryoblastic leukemia.

Chronic myelogenous leukemia (CML) occurs mainly in adults. A very small number of children also develop this disease. Treatment is with imatinib (Gleevec in US, Glivec in Europe) or other drugs. The five-year survival rate is 90%. One subtype is chronic monocytic leukemia.

Hairy cell leukemia (HCL) is sometimes considered a subset of CLL, but does not fit neatly into this pattern. About 80% of affected people are adult men. There are no reported cases in young children. HCL is incurable, but easily treatable. Survival is 96% to 100% at ten years.

T-cell prolymphocytic leukemia (T-PLL) is a very rare and aggressive leukemia affecting adults; somewhat more men than women are diagnosed with this disease. Despite its overall rarity, it is also the most common type of mature T cell leukemia; nearly all other leukemias involve B cells. It is difficult to treat, and the median survival is measured in months.

Large granular lymphocytic leukemia may involve either T-cells or NK cells; like hairy cell leukemia, which involves solely B cells, it is a rare and indolent (not aggressive) leukemia.

Adult T-cell leukemia is caused by human T-lymphotropic virus (HTLV), a virus similar to HIV. Like HIV, HTLV infects CD4+ T-cells and replicates within them; however, unlike HIV, it does not destroy them. Instead, HTLV “immortalizes” the infected T-cells, giving them the ability to proliferate abnormally.

Damage to the bone marrow, by way of displacing the normal bone marrow cells with higher numbers of immature white blood cells, results in a lack of blood platelets, which are important in the blood clotting process. This means people with leukemia may easily become bruised, bleed excessively, or develop pinprick bleeds (petechiae). White blood cells, which are involved in fighting pathogens, may be suppressed or dysfunctional. This could cause the patient's immune system to be unable to fight off a simple infection or to start attacking other body cells. Because leukemia prevents the immune system from working normally, some patients experience frequent infection, ranging from infected tonsils, sores in the mouth, or diarrhea to life-threatening pneumonia or opportunistic infections. Finally, the red blood cell deficiency leads to anemia, which may cause dyspnea and pallor.

Some patients experience other symptoms, such as feeling sick, having fevers, chills, night sweats and other flu-like symptoms, or feeling fatigued. Some patients experience nausea or a feeling of fullness due to an enlarged liver and spleen; this can result in unintentional weight loss. If the leukemic cells invade the central nervous system, then neurological symptoms (notably headaches) can occur. All symptoms associated with leukemia can be attributed to other diseases. Consequently, leukemia is always diagnosed through medical tests.

The word leukemia, which means “white blood,” is derived from the disease's namesake high white blood cell counts that most leukemia patients have before treatment. The high number of white blood cells are apparent when a blood sample is viewed under a microscope. Frequently, these extra white blood cells are immature or dysfunctional. The excessive number of cells can also interfere with the level of other cells, causing a harmful imbalance in the blood count. Some leukemia patients do not have high white blood cell counts visible during a regular blood count. This less-common condition is called aleukemia. The bone marrow still contains cancerous white blood cells which disrupt the normal production of blood cells, but they remain in the marrow instead of entering the bloodstream, where they would be visible in a blood test. For an aleukemic patient, the white blood cell counts in the bloodstream can be normal or low. Aleukemia can occur in any of the four major types of leukemia, and is particularly common in hairy cell leukemia.

Diagnosis is usually based on repeated complete blood counts and a bone marrow examination following observations of the symptoms, however, in rare cases blood tests may not show if a patient has leukemia, usually this is because the leukemia is in the early stages or has entered remission. A lymph node biopsy can be performed as well in order to diagnose certain types of leukemia in certain situations.

Following diagnosis, blood chemistry tests can be used to determine the degree of liver and kidney damage or the effects of chemotherapy on the patient. When concerns arise about visible damage due to leukemia, doctors may use an X-ray, MRI, or ultrasound. These can potentially view leukemia's effects on such body parts as bones (X-ray), the brain (MRI), or the kidneys, spleen, and liver (ultrasound). Finally, CT scans are rarely used to check lymph nodes in the chest.

Most forms of leukemia are treated with pharmaceutical medication, typically combined into a multi-drug chemotherapy regimen. Some are also treated with radiation therapy. In some cases, a bone marrow transplant is useful.

Acute lymphoblastic. Management of ALL focuses on control of bone marrow and systemic (whole-body) disease. Additionally, treatment must prevent leukemic cells from spreading to other sites, particularly the central nervous system (CNS), e.g., monthly lumbar punctures. In general, ALL treatment is divided into several phases. Induction chemotherapy to bring about bone marrow remission. For adults, standard induction plans include prednisone, vincristine, and an anthracycline drug; other drug plans may include L-asparaginase or cyclophosphamide. For children with low-risk ALL, standard therapy usually consists of three drugs (prednisone, L-asparaginase, and vincristine) for the first month of treatment.

Consolidation therapy or intensification therapy to eliminate any remaining leukemia cells. There are many different approaches to consolidation, but it is typically a high-dose, multi-drug treatment that is undertaken for a few months. Patients with low- to average-risk ALL receive therapy with antimetabolite drugs such as methotrexate and 6-mercaptopurine (6-MP). High-risk patients receive higher drug doses of these drugs, plus additional drugs.

CNS prophylaxis (preventive therapy) to stop the cancer from spreading to the brain and nervous system in high-risk patients. Standard prophylaxis may include radiation of the head and/or drugs delivered directly into the spine. Maintenance treatments with chemotherapeutic drugs to prevent disease recurrence once remission has been achieved. Maintenance therapy usually involves lower drug doses, and may continue for up to three years. Alternatively, allogeneic bone marrow transplantation may be appropriate for high-risk or relapsed patients.

Chronic lymphocytic. Hematologists base CLL treatment on both the stage and symptoms of the individual patient. A large group of CLL patients have low-grade disease, which does not benefit from treatment. Individuals with CLL-related complications or more advanced disease often benefit from treatment. In general, the indications for treatment are:
falling hemoglobin or platelet count
progression to a later stage of disease
painful, disease-related overgrowth of lymph nodes or spleen
an increase in the rate of lymphocyte production
   CLL is probably incurable by present treatments. The primary chemotherapeutic plan is combination chemotherapy with chlorambucil or cyclophosphamide, plus a corticosteroid such as prednisone or prednisolone. The use of a corticosteroid has the additional benefit of suppressing some related autoimmune diseases, such as immunohemolytic anemia or immune-mediated thrombocytopenia. In resistant cases, single-agent treatments with nucleoside drugs such as fludarabine, pentostatin, or cladribine may be successful. Younger patients may consider allogeneic or autologous bone marrow transplantation.

Acute myelogenous. Many different anti-cancer drugs are effective for the treatment of AML. Treatments vary somewhat according to the age of the patient and according to the specific subtype of AML. Overall, the strategy is to control bone marrow and systemic (whole-body) disease, while offering specific treatment for the central nervous system (CNS), if involved. In general, most oncologists rely on combinations of drugs for the initial, induction phase of chemotherapy. Such combination chemotherapy usually offers the benefits of early remission and a lower risk of disease resistance. Consolidation and maintenance treatments are intended to prevent disease recurrence. Consolidation treatment often entails a repetition of induction chemotherapy or the intensification chemotherapy with additional drugs. By contrast, maintenance treatment involves drug doses that are lower than those administered during the induction phase.

Chronic myelogenous. There are many possible treatments for CML, but the standard of care for newly diagnosed patients is imatinib (Gleevec) therapy. Compared to most anti-cancer drugs, it has relatively few side effects and can be taken orally at home. With this drug, more than 90% of patients will be able to keep the disease in check for at least five years, so that CML becomes a chronic, manageable condition. In a more advanced, uncontrolled state, when the patient cannot tolerate imatinib, or if the patient wishes to attempt a permanent cure, then an allogeneic bone marrow transplantation may be performed. This procedure involves high-dose chemotherapy and radiation followed by infusion of bone marrow from a compatible donor. Approximately 30% of patients die from this procedure.

Hairy cell. Patients with hairy cell leukemia who are symptom-free typically do not receive immediate treatment. Treatment is generally considered necessary when the patient shows signs and symptoms such as low blood cell counts (e.g., infection-fighting neutrophil count below 1.0 K/μL), frequent infections, unexplained bruises, anemia, or fatigue that is significant enough to disrupt the patient's everyday life.

Patients who need treatment usually receive either one week of cladribine, given daily by intravenous infusion or a simple injection under the skin, or six months of pentostatin, given every four weeks by intravenous infusion. In most cases, one round of treatment will produce a prolonged remission. Other treatments include rituximab infusion or self-injection with Interferon-alpha. In limited cases, the patient may benefit from splenectomy (removal of the spleen). These treatments are not typically given as the first treatment because their success rates are lower than cladribine or pentostatin.

T-cell prolymphocytic. Most patients with T-cell prolymphocytic leukemia, a rare and aggressive leukemia with a median survival of less than one year, require immediate treatment. T-cell prolymphocytic leukemia is difficult to treat, and it does not respond to most available chemotherapeutic drugs. Many different treatments have been attempted, with limited success in certain patients: purine analogues (pentostatin, fludarabine, cladribine), chlorambucil, and various forms of combination chemotherapy (cyclophosphamide, doxorubicin, vincristine, prednisone CHOP, cyclophosphamide, vincristine, prednisone [COP], vincristine, doxorubicin, prednisone, etoposide, cyclophosphamide, bleomycin VAPEC-B). Alemtuzumab (Campath), a monoclonal antibody that attacks white blood cells, has been used in treatment with greater success than previous options. Some patients who successfully respond to treatment also undergo stem cell transplantation to consolidate the response.

Juvenile myelomonocytic. Treatment for juvenile myelomonocytic leukemia can include splenectomy, chemotherapy, and bone marrow transplantation.

### II. Cancer Markers

In accordance with the methods described in greater detail in the following Examples, the inventor has identified 4- and 10-gene signatures for predicting outcomes and staging of various ERG-related cancers. The 4-marker set includes ING3, LEF1, FRZB, ANXA4, and the 10-marker set includes ING3, LEF1, MEIS2, FRZB, ANXA4, ANK3, CHD5, PLA2G7, WNT2 and SDCBP. Of these, ING3, LEF1, SDCBP, FRZB and ANXA4 are underexpressed in aggressive cancer tissues, while ANK3, CHD5, PLA2G7, WNT2 and MEIS2 are overexpressed in these tissues. When further considering PTEN status, SDCBP and MEIS2 switch and become over- and under-expressed, respectively.

In order to understand and investigate the association between the 10-gene signature and ERG rearrangement, the inventor used STRING 8.3 (Interrogation of Molecular Pathways Involved in Prostate Cancer Progression in relation to ERG rearrangements) (Szklarczyk et al., 2011) to identify genes/proteins that functionally interact with the 10-gene signature. Literature-based interactions were omitted. The analysis showed that LEF1, SRC, PITX2 and β-catenin connected all the genes in the network; however, adding these to the 10-gene signature did not improve the ERG-positive classification performance of the model. A possible explanation is that these connecting genes may not be differentially expressed, but they rather play a role in regulating expression of the other 10 genes.

To further characterize the biological processes altered or deregulated as a result of ERG rearrangement, the inventor analyzed the 10-gene signature in addition to SRC, PITX2, and β-catenin with DAVID 6.740 to identify gene ontology (GO) and pathway enrichment, and the inventor used Enrichment Map (Merico et al., 2010) to represent the enriched terms. Serveral pathway enrichment terms were identified for biological processes including: gene expression regulation and RNA metabolic processes; E-cadherin signaling and regulation of epithelial-to-mesenchymal transition; SMAD proteins and TGF-B signaling pathway; syndecan activity; and regulation of the WNT signaling pathway that has been showed to be very important in ERG mediated prostate progression (Tomlins et al., 2011). DAVID analysis also suggested that the ERG-related 10-gene signature may also be involved in other types of cancers, such as pancreatic, thyroid, colorectal, endometrial, prostate and melanogenesis.

Integrating biological networks into biomarker discovery has gained popularity recently because almost all biomarker discovery methods identify passenger biomarkers do not necessarily account for driver biomarkers, or the mechanisms that drive changes in gene expression of the identified. The inventor used network biology to identify genes that are highly associated with the biomarker set to give us a clearer picture of the biological processes and pathways in which these genes play a role. They found that these markers were associated with SRC and APC signalling pathways. APC acts as an antagonist of the Wnt signaling pathway and is also involved in other processes including cell migration and adhesion. Interestingly, several of the genes in this marker set are directly related to Wnt signalling (i.e., LEF1, FRZB, and WNT2), as is β-catenin, one of the connecting genes identified. PITX2 and SMAD (Tomlins et al., 2006; Esgueva et al., 2010) are hub genes in the biological network and play role in triggering and mediating gene expression; PITX2 is a transcription factor and SMAD is a coactivator and mediator of signal transduction by TGF-beta. Integrating biological networks with the traditional bioinformatic analysis revealed that this marker list is highly associated with driver biomarkers that play multiple varying roles in cancer development and progression.

These findings suggest that the combinational approach for biomarker discovery is a powerful one, and allows for the identification of both primary (passenger) biomarkers as well as driving markers or pathways associated with them. With development, these and other sets of biomarkers may be able to accurately assess the stage or state of the disease in a patient, or predict the severity of a disease from early stages. In addition, identification of biological pathways through these sets of biomarkers will allow us to perform further investigations into the mechanisms behind ERG-related cancer development and progression.

It is within the general scope of the present invention to provide methods for the detection of mRNA and proteins from the list above. Any method of detection known to one of skill in the art falls within the general scope of the present invention.

A. Nucleic Acid Detection

Nucleic acid sequences disclosed herein will find use in detecting expression of target genes, e.g., as probes or primers for embodiments involving nucleic acid hybridization. As used in this application, the term “polynucleotide” refers to a nucleic acid molecule that has been isolated essentially or substantially free of total genomic nucleic acid to permit hybridization and amplification, but is not limited to such. An oligonucleotide refers to a nucleic acid molecule that is complementary or identical to at least 5 contiguous nucleotides of a given sequence.

It also is contemplated that a particular polypeptide from a given species may be represented by natural variants that have slightly different nucleic acid sequences but, nonetheless, encode the same protein. In this respect, the term “gene” is used for simplicity to refer to a functional protein, polypeptide, or peptide-encoding unit. As will be understood by those in the art, this functional term includes genomic sequences, cDNA sequences, and smaller engineered gene segments that express, or may be adapted to express, proteins, polypeptides, domains, peptides, fusion proteins, and mutants.

A nucleic acid may be of the following lengths: about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1095, 1100, 1500, 2000, 2500, 3000, 3500, 4000, 4500, 5000, 5500, 6000, 6500, 7000, 7500, 8000, 9000, 10000, or more nucleotides, nucleosides, or base pairs.

1. Hybridization

The use of a probe or primer of between 13 and 100 nucleotides, preferably between 17 and 100 nucleotides in length, or in some aspects of the invention up to 1-2 kilobases or more in length, allows the formation of a duplex molecule that is both stable and selective. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

Accordingly, the nucleotide sequences of the invention may be used for their ability to selectively form duplex molecules with complementary stretches of DNAs and/or RNAs or to provide primers for amplification of DNA or RNA from samples. Depending on the application envisioned, one would desire to employ varying conditions of hybridization to achieve varying degrees of selectivity of the probe or primers for the target sequence.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications it is appreciated that lower stringency conditions are preferred. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples.

In general, it is envisioned that the probes or primers described herein will be useful as reagents in solution hybridization, as in PCR™, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772 and U.S. Patent Publication 2008/0009439. The relevant portions of these and other references identified in this section of the Specification are incorporated herein by reference.

2. In Situ Hybrization

In situ hybridization (ISH) is a type of hybridization that uses a labeled complementary DNA or RNA strand (i.e., probe) to localize a specific DNA or RNA sequence in a portion or section of tissue (in situ), or, if the tissue is small enough (e.g. plant seeds, Drosophila embryos), in the entire tissue (whole mount ISH). This is distinct from immunohistochemistry, which localizes proteins in tissue sections. Fluorescent DNA ISH (FISH) can, for example, be used in medical diagnostics to assess chromosomal integrity. RNA ISH (hybridization histochemistry) is used to measure and localize mRNAs and other transcripts within tissue sections or whole mounts.

For hybridization histochemistry, sample cells and tissues are usually treated to fix the target transcripts in place and to increase access of the probe. As noted above, the probe is either a labeled complementary DNA or, now most commonly, a complementary RNA (riboprobe). The probe hybridizes to the target sequence at elevated temperature, and then the excess probe is washed away (after prior hydrolysis using RNase in the case of unhybridized, excess RNA probe). Solution parameters such as temperature, salt and/or detergent concentration can be manipulated to remove any non-identical interactions (i.e., only exact sequence matches will remain bound). Then, the probe that was labeled with either radio-, fluorescent- or antigen-labeled bases (e.g., digoxigenin) is localized and quantitated in the tissue using either autoradiography, fluorescence microscopy or immunohistochemistry, respectively. ISH can also use two or more probes, labeled with radioactivity or the other non-radioactive labels, to simultaneously detect two or more transcripts.

3. Amplification of Nucleic Acids

Nucleic acids used as a template for amplification may be isolated from cells, tissues or other samples according to standard methodologies (Sambrook et al., 2001). In certain embodiments, analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to any sequence corresponding to a nucleic acid sequence are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids containing one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemiluminescence, radioactive scintigraphy of incorporated radiolabel or fluorescent label or even via a system using electrical and/or thermal impulse signals (Bellus, 1994).

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1988, each of which is incorporated herein by reference in their entirety.

A reverse transcriptase PCR™ amplification procedure may be performed to quantify the amount of mRNA amplified. Methods of reverse transcribing RNA into cDNA are well known (see Sambrook et al., 2001). Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864.

Reverse transcription (RT) of RNA to cDNA followed by quantitative PCR (RT-PCR) can be used to determine the relative concentrations of specific mRNA species isolated from a cell. By determining that the concentration of a specific mRNA species varies, it is shown that the gene encoding the specific mRNA species is differentially expressed. If a graph is plotted in which the cycle number is on the X axis and the log of the concentration of the amplified target DNA is on the Y axis, a curved line of characteristic shape is formed by connecting the plotted points. Beginning with the first cycle, the slope of the line is positive and constant. This is said to be the linear portion of the curve. After a reagent becomes limiting, the slope of the line begins to decrease and eventually becomes zero. At this point the concentration of the amplified target DNA becomes asymptotic to some fixed value. This is said to be the plateau portion of the curve.

The concentration of the target DNA in the linear portion of the PCR amplification is directly proportional to the starting concentration of the target before the reaction began. By determining the concentration of the amplified products of the target DNA in PCR reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific mRNA from which the target sequence was derived can be determined for the respective tissues or cells. This direct proportionality between the concentration of the PCR products and the relative mRNA abundances is only true in the linear range of the PCR reaction.

The final concentration of the target DNA in the plateau portion of the curve is determined by the availability of reagents in the reaction mix and is independent of the original concentration of target DNA. Therefore, the first condition that must be met before the relative abundances of a mRNA species can be determined by RT-PCR for a collection of RNA populations is that the concentrations of the amplified PCR products must be sampled when the PCR reactions are in the linear portion of their curves.

A second condition for an RT-PCR experiment is to determine the relative abundances of a particular mRNA species. Typically, relative concentrations of the amplifiable cDNAs are normalized to some independent standard. The goal of an RT-PCR experiment is to determine the abundance of a particular mRNA species relative to the average abundance of all mRNA species in the sample.

Most protocols for competitive PCR utilize internal PCR standards that are approximately as abundant as the target. These strategies are effective if the products of the PCR amplifications are sampled during their linear phases. If the products are sampled when the reactions are approaching the plateau phase, then the less abundant product becomes relatively over represented. Comparisons of relative abundances made for many different RNA samples, such as is the case when examining RNA samples for differential expression, become distorted in such a way as to make differences in relative abundances of RNAs appear less than they actually are. This is not a significant problem if the internal standard is much more abundant than the target. If the internal standard is more abundant than the target, then direct linear comparisons can be made between RNA samples.

RT-PCR can be performed as a relative quantitative RT-PCR with an internal standard in which the internal standard is an amplifiable cDNA fragment that is larger than the target cDNA fragment and in which the abundance of the mRNA encoding the internal standard is roughly 5-100 fold higher than the mRNA encoding the target. This assay measures relative abundance, not absolute abundance of the respective mRNA species.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assy (OLA), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence based amplification (NASBA) and 3SR (Kwoh et al., 1989; PCT Application WO 88/10315, incorporated herein by reference in their entirety). European Application No. 329 822 disclose a nucleic acid amplification process involving cyclically synthesizing single-stranded RNA (“ssRNA”), ssDNA, and double-stranded DNA (dsDNA), which may be used in accordance with the present invention.

PCT Application WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR” (Frohman, 1990; Ohara et al., 1989).

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 2001). Separated amplification products may be cut out and eluted from the gel for further manipulation. Using low melting point agarose gels, the separated band may be removed by heating the gel, followed by extraction of the nucleic acid.

Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art (see Sambrook et al., 2001). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention. Various nucleic acid detection methods known in the art are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

4. Gene Methylation

DNA methylation is a biochemical process that is important for normal development in higher organisms. It involves the addition of a methyl group to the 5 position of the cytosine pyrimidine ring or the number 6 nitrogen of the adenine purine ring. This modification can be inherited through cell division. DNA methylation is a crucial part of normal organismal development and cellular differentiation in higher organisms. DNA methylation stably alters the gene expression pattern in cells; for example, cells programmed to be pancreatic islets during embryonic development remain pancreatic islets throughout the life of the organism without continuing signals telling them that they need to remain islets. DNA methylation is typically removed during zygote formation and re-established through successive cell divisions during development. However, research shows that hydroxylation of methyl group occurs rather than complete removal of methyl groups in zygote. Some methylation modifications that regulate gene expression are inheritable and are referred to as epigenetic regulation.

In addition, DNA methylation suppresses the expression of viral genes and other deleterious elements that have been incorporated into the genome of the host over time. DNA methylation also forms the basis of chromatin structure, which enables cells to form the myriad characteristics necessary for multicellular life from a single immutable sequence of DNA. DNA methylation also plays a crucial role in the development of nearly all types of cancer. DNA methylation at the 5 position of cytosine has the specific effect of reducing gene expression and has been found in every vertebrate examined. In adult somatic tissues, DNA methylation typically occurs in a CpG dinucleotide context; non-CpG methylation is prevalent in embryonic stem cells.

DNA methylation is an important regulator of gene transcription and a large body of evidence has demonstrated that aberrant DNA methylation is associated with unscheduled gene silencing, and the genes with high levels of 5-methylcytosine in their promoter region are transcriptionally silent. DNA methylation is essential during embryonic development, and in somatic cells, patterns of DNA methylation are generally transmitted to daughter cells with a high fidelity. Aberrant DNA methylation patterns have been associated with a large number of human malignancies and found in two distinct forms: hypermethylation and hypomethylation compared to normal tissue. Hypermethylation is one of the major epigenetic modifications that repress transcription via promoter region of tumour suppressor genes. Hypermethylation typically occurs at CpG islands in the promoter region and is associated with gene inactivation. Global hypomethylation has also been implicated in the development and progression of cancer through different mechanisms.

5. Chip Technologies

Specifically contemplated by the present inventor are chip-based DNA technologies such as those described by Hacia et al. (1996) and Shoemaker et al. (1996). Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization (see also, Pease et al., 1994; and Fodor et al, 1991). It is contemplated that this technology may be used in conjunction with evaluating the expression level of a gene target.

6. Nucleic Acid Arrays

The present invention may involve the use of arrays or data generated from an array. Data may be readily available. An array generally refers to ordered macroarrays or microarrays of nucleic acid molecules (probes) that are fully or nearly complementary or identical to a plurality of mRNA molecules or cDNA molecules and that are positioned on a support material in a spatially separated organization. Macroarrays are typically sheets of nitrocellulose or nylon upon which probes have been spotted. Microarrays position the nucleic acid probes more densely such that up to 10,000 nucleic acid molecules can be fit into a region typically 1 to 4 square centimeters. Microarrays can be fabricated by spotting nucleic acid molecules, e.g., genes, oligonucleotides, etc., onto substrates or fabricating oligonucleotide sequences in situ on a substrate. Spotted or fabricated nucleic acid molecules can be applied in a high density matrix pattern of up to about 30 non-identical nucleic acid molecules per square centimeter or higher, e.g., up to about 100 or even 1000 per square centimeter. Microarrays typically use coated glass as the solid support, in contrast to the nitrocellulose-based material of filter arrays. By having an ordered array of complementing nucleic acid samples, the position of each sample can be tracked and linked to the original sample. A variety of different array devices in which a plurality of distinct nucleic acid probes are stably associated with the surface of a solid support are known to those of skill in the art. Useful substrates for arrays include nylon, glass and silicon Such arrays may vary in a number of different ways, including average probe length, sequence or types of probes, nature of bond between the probe and the array surface, e.g., covalent or non-covalent, and the like. The labeling and screening methods of the present invention and the arrays are not limited in its utility with respect to any parameter except that the probes detect expression levels; consequently, methods and compositions may be used with a variety of different types of genes.

Representative methods and apparatus for preparing a microarray have been described, for example, in U.S. Pat. Nos. 5,143,854; 5,202,231; 5,242,974; 5,288,644; 5,324,633; 5,384,261; 5,405,783; 5,412,087; 5,424,186; 5,429,807; 5,432,049; 5,436,327; 5,445,934; 5,468,613; 5,470,710; 5,472,672; 5,492,806; 5,503,980; 5,510,270; 5,525,464; 5,527,681; 5,529,756; 5,532,128; 5,545,531; 5,547,839; 5,554,501; 5,556,752; 5,561,071; 5,571,639; 5,580,726; 5,580,732; 5,593,839; 5,599,695; 5,599,672; 5,610,287; 5,624,711; 5,631,134; 5,639,603; 5,654,413; 5,658,734; 5,661,028; 5,665,547; 5,667,972; 5,695,940; 5,700,637; 5,744,305; 5,800,992; 5,807,522; 5,830,645; 5,837,196; 5,871,928; 5,847,219; 5,876,932; 5,919,626; 6,004,755; 6,087,102; 6,368,799; 6,383,749; 6,617,112; 6,638,717; 6,720,138, as well as WO 93/17126; WO 95/11995; WO 95/21265; WO 95/21944; WO 95/35505; WO 96/31622; WO 97/10365; WO 97/27317; WO 99/35505; WO 09923256; WO 09936760; WO0138580; WO 0168255; WO 03020898; WO 03040410; WO 03053586; WO 03087297; WO 03091426; WO03100012; WO 04020085; WO 04027093; EP 373 203; EP 785 280; EP 799 897 and UK 8 803 000; the disclosures of which are all herein incorporated by reference.

It is contemplated that the arrays can be high density arrays, such that they contain 100 or more different probes. It is contemplated that they may contain 1000, 16,000, 65,000, 250,000 or 1,000,000 or more different probes. The probes can be directed to targets in one or more different organisms. The oligonucleotide probes range from 5 to 50, 5 to 45, 10 to 40, or 15 to 40 nucleotides in length in some embodiments. In certain embodiments, the oligonucleotide probes are 20 to 25 nucleotides in length.

The location and sequence of each different probe sequence in the array are generally known. Moreover, the large number of different probes can occupy a relatively small area providing a high density array having a probe density of generally greater than about 60, 100, 600, 1000, 5,000, 10,000, 40,000, 100,000, or 400,000 different oligonucleotide probes per cm². The surface area of the array can be about or less than about 1, 1.6, 2, 3, 4, 5, 6, 7, 8, 9, or 10 cm².

Moreover, a person of ordinary skill in the art could readily analyze data generated using an array. Such protocols are disclosed above, and include information found in WO 9743450; WO 03023058; WO 03022421; WO 03029485; WO 03067217; WO 03066906; WO 03076928; WO 03093810; WO 03100448A1, all of which are specifically incorporated by reference.

B. Protein Detection

In certain embodiments, the present invention concerns determining the expression level of a protein corresponding to a target gene. As used herein, a “protein,” “proteinaceous molecule,” “proteinaceous composition,” “proteinaceous compound,” “proteinaceous chain” or “proteinaceous material” generally refers, but is not limited to, a protein of greater than about 200 amino acids or the full length endogenous sequence translated from a gene; a polypeptide of greater than about 100 amino acids; and/or a peptide of from about 3 to about 100 amino acids. All the “proteinaceous” terms described above may be used interchangeably herein.

In certain embodiments, the proteinaceous composition may be identified using an antibody. As used herein, the term “antibody” is intended to refer broadly to any immunologic binding agent such as IgG, IgM, IgA, IgD and IgE. The term “antibody” is used to refer to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies are also well known in the art (see, e.g., Harlow et al., 1988; incorporated herein by reference).

1. Proteinaceous Compositions

As used herein, an “amino molecule” refers to any amino acid, amino acid derivative or amino acid mimic as would be known to one of ordinary skill in the art. In certain embodiments, the residues of the proteinaceous molecule are sequential, without any non-amino molecule interrupting the sequence of amino molecule residues. In other embodiments, the sequence may comprise one or more non-amino molecule moieties. In particular embodiments, the sequence of residues of the proteinaceous molecule may be interrupted by one or more non-amino molecule moieties. Accordingly, the term “proteinaceous composition” encompasses amino molecule sequences comprising at least one of the 20 common amino acids in naturally synthesized proteins, or at least one modified or unusual amino acid.

Proteinaceous compositions may be made by any technique known to those of skill in the art, including the expression of proteins, polypeptides or peptides through standard molecular biological techniques, the isolation of proteinaceous compounds from natural sources, or the chemical synthesis of proteinaceous materials. In certain embodiments a proteinaceous compound may be purified. Generally, “purified” will refer to a specific or protein, polypeptide, or peptide composition that has been subjected to fractionation to remove various other proteins, polypeptides, or peptides, and which composition substantially retains its activity, as may be assessed, for example, by the protein assays, as would be known to one of ordinary skill in the art for the specific or desired protein, polypeptide or peptide.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or even HPLC.

The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur. Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulfate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (e.g., alter pH, ionic strength, and temperature).

A particular type of affinity chromatography useful in the purification of carbohydrate containing compounds is lectin affinity chromatography. Lectins are a class of substances that bind to a variety of polysaccharides and glycoproteins. Lectins are usually coupled to agarose by cyanogen bromide. Conconavalin A coupled to Sepharose was the first material of this sort to be used and has been widely used in the isolation of polysaccharides and glycoproteins other lectins that have been include lentil lectin, wheat germ agglutinin which has been useful in the purification of N-acetyl glucosaminyl residues and Helix pomatia lectin. Lectins themselves are purified using affinity chromatography with carbohydrate ligands. Lactose has been used to purify lectins from castor bean and peanuts; maltose has been useful in extracting lectins from lentils and jack bean; N-acetyl-D galactosamine is used for purifying lectins from soybean; N-acetyl glucosaminyl binds to lectins from wheat germ; D-galactosamine has been used in obtaining lectins from clams and L-fucose will bind to lectins from lotus.

The matrix should be a substance that by itself does not adsorb molecules to any significant extent and that has a broad range of chemical, physical and thermal stability. The ligand should be coupled in such a way as to not affect its binding properties. The ligand also should provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed below.

2. Immunodetection Methods

In some embodiments, the present invention concerns immunodetection methods. Immunodetection methods include enzyme linked immunosorbent assay (ELISA), radioimmunoassay (RIA), immunoradiometric assay, fluoroimmunoassay, chemiluminescent assay, bioluminescent assay, and Western blot, though several others are well known to those of ordinary skill. The steps of various useful immunodetection methods have been described in the scientific literature, such as, e.g., Doolittle et al. (1999); Gulbis et al. (1993); De Jager et al. (1993); and Nakamura et al. (1987), each incorporated herein by reference.

In general, the immunobinding methods include obtaining a sample containing a protein, polypeptide and/or peptide, and contacting the sample with a first antibody, monoclonal or polyclonal, in accordance with the present invention, as the case may be, under conditions effective to allow the formation of immunocomplexes.

These methods include methods for purifying a protein, polypeptide and/or peptide from organelle, cell, tissue or organism's samples. In these instances, the antibody removes the antigenic protein, polypeptide and/or peptide component from a sample. The antibody will preferably be linked to a solid support, such as in the form of a column matrix, and the sample suspected of containing the protein, polypeptide and/or peptide antigenic component will be applied to the immobilized antibody. The unwanted components will be washed from the column, leaving the antigen immunocomplexed to the immobilized antibody to be eluted.

The immunobinding methods also include methods for detecting and quantifying the amount of an antigen component in a sample and the detection and quantification of any immune complexes formed during the binding process. Here, one would obtain a sample suspected of containing an antigen or antigenic domain, and contact the sample with an antibody against the antigen or antigenic domain, and then detect and quantify the amount of immune complexes formed under the specific conditions.

In terms of antigen detection, the biological sample analyzed may be any sample that is suspected of containing an antigen or antigenic domain, such as, for example, a tissue section or specimen, a homogenized tissue extract, a cell, an organelle, separated and/or purified forms of any of the above antigen-containing compositions, or even any biological fluid that comes into contact with the cell or tissue, including blood and/or serum.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. U.S. patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection designed by Charles Cantor uses two different antibodies. A first step biotinylated, monoclonal or polyclonal antibody is used to detect the target antigen(s), and a second step antibody is then used to detect the biotin attached to the complexed biotin. In that method the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

3. ELISAs

As detailed above, immunoassays, in their most simple and/or direct sense, are binding assays. Certain preferred immunoassays are the various types of enzyme linked immunosorbent assays (ELISAs) and/or radioimmunoassays (RIA) known in the art. Immunohistochemical detection using tissue sections is also particularly useful. However, it will be readily appreciated that detection is not limited to such techniques, and/or western blotting, dot blotting, FACS analyses, and/or the like may also be used.

In one exemplary ELISA, antibodies are immobilized onto a selected surface exhibiting protein affinity, such as a well in a polystyrene microtiter plate. Then, a test composition suspected of containing the antigen, such as a clinical sample, is added to the wells. After binding and/or washing to remove non-specifically bound immune complexes, the bound antigen may be detected. Detection is generally achieved by the addition of another antibody that is linked to a detectable label. This type of ELISA is a simple “sandwich ELISA.” Detection may also be achieved by the addition of a second antibody, followed by the addition of a third antibody that has binding affinity for the second antibody, with the third antibody being linked to a detectable label.

In another exemplary ELISA, the samples suspected of containing the antigen are immobilized onto the well surface and/or then contacted with antibodies. After binding and/or washing to remove non-specifically bound immune complexes, the bound anti-antibodies are detected. Where the initial antibodies are linked to a detectable label, the immune complexes may be detected directly. Again, the immune complexes may be detected using a second antibody that has binding affinity for the first antibody, with the second antibody being linked to a detectable label.

Another ELISA in which the antigens are immobilized, involves the use of antibody competition in the detection. In this ELISA, labeled antibodies against an antigen are added to the wells, allowed to bind, and/or detected by means of their label. The amount of an antigen in an unknown sample is then determined by mixing the sample with the labeled antibodies against the antigen during incubation with coated wells. The presence of an antigen in the sample acts to reduce the amount of antibody against the antigen available for binding to the well and thus reduces the ultimate signal. This is also appropriate for detecting antibodies against an antigen in an unknown sample, where the unlabeled antibodies bind to the antigen-coated wells and also reduces the amount of antigen available to bind the labeled antibodies.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Following all incubation steps in an ELISA, the contacted surface is washed so as to remove non-complexed material. An example of a washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. This may be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

4. Immunohistochemistry

The antibodies of the present invention may also be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors, and/or is well known to those of skill in the art (Brown et al., 1990; Abbondanzo et al., 1990; Allred et al., 1990).

Immunohistochemistry or IHC refers to the process of localizing proteins in cells of a tissue section exploiting the principle of antibodies binding specifically to antigens in biological tissues. It takes its name from the roots “immuno,” in reference to antibodies used in the procedure, and “histo,” meaning tissue. Immunohistochemical staining is widely used in the diagnosis and treatment of cancer.

Visualising an antibody-antigen interaction can be accomplished in a number of ways. In the most common instance, an antibody is conjugated to an enzyme, such as peroxidase, that can catalyse a colour-producing reaction. Alternatively, the antibody can also be tagged to a fluorophore, such as FITC, rhodamine, Texas Red, Alexa Fluor, or DyLight Fluor. The latter method is of great use in confocal laser scanning microscopy, which is highly sensitive and can also be used to visualize interactions between multiple proteins.

Briefly, frozen-sections may be prepared by rehydrating 50 mg of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections.

There are two strategies used for the immunohistochemical detection of antigens in tissue, the direct method and the indirect method. In both cases, the tissue is treated to rupture the membranes, usually by using a kind of detergent called Triton X-100.

The direct method is a one-step staining method, and involves a labeled antibody (e.g. FITC conjugated antiserum) reacting directly with the antigen in tissue sections. This technique utilizes only one antibody and the procedure is therefore simple and rapid. However, it can suffer problems with sensitivity due to little signal amplification and is in less common use than indirect methods.

The indirect method involves an unlabeled primary antibody (first layer) which reacts with tissue antigen, and a labeled secondary antibody (second layer) which reacts with the primary antibody. The secondary antibody must be against the IgG of the animal species in which the primary antibody has been raised. This method is more sensitive due to signal amplification through several secondary antibody reactions with different antigenic sites on the primary antibody. The second layer antibody can be labeled with a fluorescent dye or an enzyme.

In a common procedure, a biotinylated secondary antibody is coupled with streptavidin-horseradish peroxidase. This is reacted with 3,3′-Diaminobenzidine (DAB) to produce a brown staining wherever primary and secondary antibodies are attached in a process known as DAB staining. The reaction can be enhanced using nickel, producing a deep purple/gray staining.

The indirect method, aside from its greater sensitivity, also has the advantage that only a relatively small number of standard conjugated (labeled) secondary antibodies needs to be generated. For example, a labeled secondary antibody raised against rabbit IgG, which can be purchased “off the shelf,” is useful with any primary antibody raised in rabbit. With the direct method, it would be necessary to make custom labeled antibodies against every antigen of interest.

5. Protein Arrays

Protein array technology is discussed in detail in Pandey and Mann (2000) and MacBeath and Schreiber (2000), each of which is herein specifically incorporated by reference.

These arrays, typcially contain thousands of different proteins or antibodies spotted onto glass slides or immobilized in tiny wells, allow one to examine the biochemical activities and binding profiles of a large number of proteins at once. To examine protein interactions with such an array, a labeled protein is incubated with each of the target proteins immobilized on the slide, and then one determines which of the many proteins the labeled molecule binds. In certain embodiments such technology can be used to quantitate a number of proteins in a sample.

The basic construction of protein chips has some similarities to DNA chips, such as the use of a glass or plastic surface dotted with an array of molecules. These molecules can be DNA or antibodies that are designed to capture proteins. Defined quantities of proteins are immobilized on each spot, while retaining some activity of the protein. With fluorescent markers or other methods of detection revealing the spots that have captured these proteins, protein microarrays are being used as powerful tools in high-throughput proteomics and drug discovery.

The earliest and best-known protein chip is the ProteinChip by Ciphergen Biosystems Inc. (Fremont, Calif.). The ProteinChip is based on the surface-enhanced laser desorption and ionization (SELDI) process. Known proteins are analyzed using functional assays that are on the chip. For example, chip surfaces can contain enzymes, receptor proteins, or antibodies that enable researchers to conduct protein-protein interaction studies, ligand binding studies, or immunoassays. With state-of-the-art ion optic and laser optic technologies, the ProteinChip system detects proteins ranging from small peptides of less than 1000 Da up to proteins of 300 kDa and calculates the mass based on time-of-flight (TOF).

The ProteinChip biomarker system is the first protein biochip-based system that enables biomarker pattern recognition analysis to be done. This system allows researchers to address important clinical questions by investigating the proteome from a range of crude clinical samples (i.e., laser capture microdissected cells, biopsies, tissue, urine, and serum).

The system also utilizes biomarker pattern software that automates pattern recognition-based statistical analysis methods to correlate protein expression patterns from clinical samples with disease phenotypes.

### III. Treatment of ERG-Related Cancer

In some embodiments, the invention further provides treatment of ERG-related cancer. One of skill in the art will be aware of many treatments and treatment combinations may be used, some but not all of which are described below.

A. Formulations and Routes for Administration to Patients

Where clinical applications are contemplated, it will be necessary to prepare pharmaceutical compositions in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the vector to cells, dissolved or dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present invention, its use in therapeutic compositions is contemplated. Supplementary active ingredients also can be incorporated into the compositions.

The active compositions of the present invention may include classic pharmaceutical preparations. Administration of these compositions according to the present invention will be via any common route so long as the target tissue is available via that route. This includes oral, nasal, buccal, rectal, vaginal or topical. Alternatively, administration may be by intradermal, subcutaneous, intramuscular, intraperitoneal or intravenous injection. Such compositions would normally be administered as pharmaceutically acceptable compositions. Of particular interest is direct intratumoral administration, perfusion of a tumor, or administration local or regional to a tumor, for example, in the local or regional vasculature or lymphatic system, or in a resected tumor bed (e.g., post-operative catheter). For practically any tumor, systemic delivery also is contemplated. This will prove especially important for attacking microscopic or metastatic cancer.

The active compounds may also be administered as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The actual dosage amount of a composition of the present invention administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

“Treatment” and “treating” refer to administration or application of a therapeutic agent to a subject or performance of a procedure or modality on a subject for the purpose of obtaining a therapeutic benefit of a disease or health-related condition.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease.

A “disease” can be any pathological condition of a body part, an organ, or a system resulting from any cause, such as infection, genetic defect, and/or environmental stress.

“Prevention” and “preventing” are used according to their ordinary and plain meaning to mean “acting before” or such an act. In the context of a particular disease, those terms refer to administration or application of an agent, drug, or remedy to a subject or performance of a procedure or modality on a subject for the purpose of blocking the onset of a disease or health-related condition.

The subject can be a subject who is known or suspected of being free of a particular disease or health-related condition at the time the relevant preventive agent is administered. The subject, for example, can be a subject with no known disease or health-related condition (i.e., a healthy subject). In additional embodiments of the invention, methods include identifying a patient in need of treatment. A patient may be identified, for example, based on taking a patient history or based on findings on clinical examination.

B. Cancer Treatments

1. Chemotherapy

A wide variety of chemotherapeutic agents may be used in accordance with the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP 16), tamoxifen, raloxifene, estrogen receptor binding agents, taxol, paclitaxel, docetaxel, gemcitabien, navelbine, farnesyl-protein tansferase inhibitors, transplatinum, 5-fluorouracil, vincristin, vinblastin and methotrexate and pharmaceutically acceptable salts, acids or derivatives of any of the above.

2. Radiotherapy

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly.

Radiation therapy used according to the present invention may include, but is not limited to, the use of γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

Radiotherapy may comprise the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells.

Conformal radiotherapy uses the same radiotherapy machine, a linear accelerator, as the normal radiotherapy treatment but metal blocks are placed in the path of the x-ray beam to alter its shape to match that of the cancer. This ensures that a higher radiation dose is given to the tumor. Healthy surrounding cells and nearby structures receive a lower dose of radiation, so the possibility of side effects is reduced. A device called a multi-leaf collimator has been developed and can be used as an alternative to the metal blocks. The multi-leaf collimator consists of a number of metal sheets which are fixed to the linear accelerator. Each layer can be adjusted so that the radiotherapy beams can be shaped to the treatment area without the need for metal blocks. Precise positioning of the radiotherapy machine is very important for conformal radiotherapy treatment and a special scanning machine may be used to check the position of your internal organs at the beginning of each treatment.

High-resolution intensity modulated radiotherapy also uses a multi-leaf collimator. During this treatment the layers of the multi-leaf collimator are moved while the treatment is being given. This method is likely to achieve even more precise shaping of the treatment beams and allows the dose of radiotherapy to be constant over the whole treatment area.

Although research studies have shown that conformal radiotherapy and intensity modulated radiotherapy may reduce the side effects of radiotherapy treatment, it is possible that shaping the treatment area so precisely could stop microscopic cancer cells just outside the treatment area being destroyed. This means that the risk of the cancer coming back in the future may be higher with these specialized radiotherapy techniques.

Scientists also are looking for ways to increase the effectiveness of radiation therapy. Two types of investigational drugs are being studied for their effect on cells undergoing radiation. Radiosensitizers make the tumor cells more likely to be damaged, and radioprotectors protect normal tissues from the effects of radiation. Hyperthermia, the use of heat, is also being studied for its effectiveness in sensitizing tissue to radiation.

3. Immunotherapy

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, γ-IFN, chemokines such as MIP-1, MCP-1, IL-8 and growth factors such as FLT3 ligand. Combining immune stimulating molecules, either as proteins or using gene delivery in combination with a tumor suppressor has been shown to enhance antitumor effects (Ju et al., 2000). Moreover, antibodies against any of these compounds can be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides et al., 1998), cytokine therapy, e.g., interferons α, β, and γ; IL-1, GM-CSF and TNF (Bukowski et al., 1998; Davidson et al., 1998; Hellstrand et al., 1998) gene therapy, e.g., TNF, IL-1, IL-2, p53 (Qin et al., 1998; Austin-Ward and Villaseca, 1998; U.S. Pat. Nos. 5,830,880 and 5,846,945) and monoclonal antibodies, e.g., anti-ganglioside GM2, anti-HER-2, anti-p185 (Pietras et al., 1998; Hanibuchi et al., 1998; U.S. Pat. No. 5,824,311). It is contemplated that one or more anti-cancer therapies may be employed with the gene silencing therapies described herein.

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton et al., 1992; Mitchell et al., 1990; Mitchell et al., 1993).

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989).

4. Surgery

Approximately 60% of persons with cancer will undergo surgery of some type, which includes preventative, diagnostic or staging, curative, and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Upon excision of part or all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Gene Therapy

In yet another embodiment, the gene therapy may be applied to the subject. Suitable genes included inducers of cellular proliferation, tumor suppressors, or regulators of programmed cell death.

6. RNA Interference (RNAi)

RNA interference (also referred to as “RNA-mediated interference” or RNAi) is a mechanism by which gene expression can be reduced or eliminated. Double-stranded RNA (dsRNA) has been observed to mediate the reduction, which is a multi-step process. dsRNA activates post-transcriptional gene expression surveillance mechanisms that appear to function to defend cells from virus infection and transposon activity (Fire et al., 1998; Grishok et al., 2000; Ketting et al., 1999; Lin and Avery et al., 1999; Montgomery et al., 1998; Sharp and Zamore, 2000; Tabara et al., 1999). Activation of these mechanisms targets mature, dsRNA-complementary mRNA for destruction. RNAi offers major experimental advantages for study of gene function. These advantages include a very high specificity, ease of movement across cell membranes, and prolonged down-regulation of the targeted gene (Fire et al., 1998; Grishok et al., 2000; Ketting et al., 1999; Lin and Avery et al., 1999; Montgomery et al., 1998; Sharp et al., 1999; Sharp and Zamore, 2000; Tabara et al., 1999). It is generally accepted that RNAi acts post-transcriptionally, targeting RNA transcripts for degradation. It appears that both nuclear and cytoplasmic RNA can be targeted (Bosher and Labouesse, 2000).

siRNAs must be designed so that they are specific and effective in suppressing the expression of the genes of interest. Methods of selecting the target sequences, i.e., those sequences present in the gene or genes of interest to which the siRNAs will guide the degradative machinery, are directed to avoiding sequences that may interfere with the siRNA's guide function while including sequences that are specific to the gene or genes. Typically, siRNA target sequences of about 21 to 23 nucleotides in length are most effective. This length reflects the lengths of digestion products resulting from the processing of much longer RNAs as described above (Montgomery et al., 1998). siRNA are well known in the art. For example, siRNA and double-stranded RNA have been described in U.S. Pat. Nos. 6,506,559 and 6,573,099, as well as in U.S. Patent Applications 2003/0051263, 2003/0055020, 2004/0265839, 2002/0168707, 2003/0159161, and 2004/0064842, all of which are herein incorporated by reference in their entirety.

Several further modifications to siRNA sequences have been suggested in order to alter their stability or improve their effectiveness. It is suggested that synthetic complementary 21-mer RNAs having di-nucleotide overhangs (i.e., 19 complementary nucleotides+3′ non-complementary dimers) may provide the greatest level of suppression. These protocols primarily use a sequence of two (2′-deoxy)thymidine nucleotides as the di-nucleotide overhangs. These dinucleotide overhangs are often written as dTdT to distinguish them from the typical nucleotides incorporated into RNA. The literature has indicated that the use of dT overhangs is primarily motivated by the need to reduce the cost of the chemically synthesized RNAs. It is also suggested that the dTdT overhangs might be more stable than UU overhangs, though the data available shows only a slight (<20%) improvement of the dTdT overhang compared to an siRNA with a UU overhang.

dsRNA can be synthesized using well-described methods (Fire et al., 1998). Briefly, sense and antisense RNA are synthesized from DNA templates using T7 polymerase (MEGAscript, Ambion). After the synthesis is complete, the DNA template is digested with DnaseI and RNA purified by phenol/chloroform extraction and isopropanol precipitation. RNA size, purity and integrity are assayed on denaturing agarose gels. Sense and antisense RNA are diluted in potassium citrate buffer and annealed at 80° C. for 3 min to form dsRNA. As with the construction of DNA template libraries, a procedures may be used to aid this time intensive procedure. The sum of the individual dsRNA species is designated as a “dsRNA library.”

The making of siRNAs has been mainly through direct chemical synthesis; through processing of longer, double-stranded RNAs through exposure to Drosophila embryo lysates; or through an in vitro system derived from S2 cells. Use of cell lysates or in vitro processing may further involve the subsequent isolation of the short, 21-23 nucleotide siRNAs from the lysate, etc., making the process somewhat cumbersome and expensive. Chemical synthesis proceeds by making two single-stranded RNA-oligomers followed by the annealing of the two single-stranded oligomers into a double-stranded RNA. Methods of chemical synthesis are diverse. Non-limiting examples are provided in U.S. Pat. Nos. 5,889,136, 4,415,723, and 4,458,066, expressly incorporated herein by reference, and in Wincott et al. (1995).

WO 99/32619 and WO 01/68836 suggest that RNA for use in siRNA may be chemically or enzymatically synthesized. Both of these texts are incorporated herein in their entirety by reference. The enzymatic synthesis contemplated in these references is by a cellular RNA polymerase or a bacteriophage RNA polymerase (e.g., T3, T7, SP6) via the use and production of an expression construct as is known in the art. For example, see U.S. Pat. No. 5,795,715. The contemplated constructs provide templates that produce RNAs that contain nucleotide sequences identical to a portion of the target gene. The length of identical sequences provided by these references is at least 25 bases, and may be as many as 400 or more bases in length. An important aspect of this reference is that the authors contemplate digesting longer dsRNAs to 21-25mer lengths with the endogenous nuclease complex that converts long dsRNAs to siRNAs in vivo. They do not describe or present data for synthesizing and using in vitro transcribed 21-25mer dsRNAs. No distinction is made between the expected properties of chemical or enzymatically synthesized dsRNA in its use in RNA interference.

Similarly, WO 00/44914, incorporated herein by reference, suggests that single strands of RNA can be produced enzymatically or by partial/total organic synthesis. Preferably, single-stranded RNA is enzymatically synthesized from the PCR products of a DNA template, preferably a cloned cDNA template and the RNA product is a complete transcript of the cDNA, which may comprise hundreds of nucleotides. WO 01/36646, incorporated herein by reference, places no limitation upon the manner in which the siRNA is synthesized, providing that the RNA may be synthesized in vitro or in vivo, using manual and/or automated procedures. This reference also provides that in vitro synthesis may be chemical or enzymatic, for example using cloned RNA polymerase (e.g., T3, T7, SP6) for transcription of the endogenous DNA (or cDNA) template, or a mixture of both. Again, no distinction in the desirable properties for use in RNA interference is made between chemically or enzymatically synthesized siRNA.

U.S. Pat. No. 5,795,715 reports the simultaneous transcription of two complementary DNA sequence strands in a single reaction mixture, wherein the two transcripts are immediately hybridized. The templates used are preferably of between 40 and 100 base pairs, and which is equipped at each end with a promoter sequence. The templates are preferably attached to a solid surface. After transcription with RNA polymerase, the resulting dsRNA fragments may be used for detecting and/or assaying nucleic acid target sequences.

Several groups have developed expression vectors that continually express siRNAs in stably transfected mammalian cells (Brummelkamp et al., 2002; Lee et al., 2002; Paul et al., 2002; Sui et al., 2002; Yu et al., 2002). Some of these plasmids are engineered to express shRNAs lacking poly (A) tails (Brummelkamp et al., 2002; Paul et al., 2002; Yu et al., 2002). Transcription of shRNAs is initiated at a polymerase III (pol III) promoter and is believed to be terminated at position 2 of a 4-5-thymine transcription termination site, shRNAs are thought to fold into a stem-loop structure with 3′ UU-overhangs. Subsequently, the ends of these shRNAs are processed, converting the shRNAs into ˜21 nt siRNA-like molecules (Brummelkamp et al., 2002). The siRNA-like molecules can, in turn, bring about gene-specific silencing in the transfected mammalian cells.

7. Other Agents

It is contemplated that other agents may be used with the present invention. These additional agents include immunomodulatory agents, agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL (Apo-2 ligand) would potentiate the apoptotic inducing abilities of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyerproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

There have been many advances in the therapy of cancer following the introduction of cytotoxic chemotherapeutic drugs. However, one of the consequences of chemotherapy is the development/acquisition of drug-resistant phenotypes and the development of multiple drug resistance. The development of drug resistance remains a major obstacle in the treatment of such tumors and therefore, there is an obvious need for alternative approaches such as gene therapy.

Another form of therapy for use in conjunction with chemotherapy, radiation therapy or biological therapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

C. Dosage

The amount of therapeutic agent to be included in the compositions or applied in the methods set forth herein will be whatever amount is pharmaceutically effective and will depend upon a number of factors, including the identity and potency of the chosen therapeutic agent. One of ordinary skill in the art would be familiar with factors that are involved in determining a therapeutically effective dose of a particular agent. Thus, in this regards, the concentration of the therapeutic agent in the compositions set forth herein can be any concentration. In some particular embodiments, the total concentration of the drug is less than 10%. In more particular embodiments, the concentration of the drug is less than 5%. The therapeutic agent may be applied once or more than once. In non-limiting examples, the therapeutic agent is applied once a day, twice a day, three times a day, four times a day, six times a day, every two hours when awake, every four hours, every other day, once a week, and so forth. Treatment may be continued for any duration of time as determined by those of ordinary skill in the art.

### IV. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Patient cohorts and Samples. The patient cohorts consisted of two cohorts, the first consists of a 61 patinets. Samples included benign, localized cancer, and castration resistant prostate cancer (26 benign, 30 localized PCA and 30 castration resistant PCA (CRPC)) assembled on one tissue microarray (TMA) using two to ten 0.6 mm cores per sample with a total of 320 cores belonging to 86 patients. This cohort was used to validate marker association to ERG and to disease progression. A second cohort of 52 CRPC patients (54 samples) was used for the interrogation of ERG status and for RNA extraction to be utilized for gene expression profilling. Tissue samples were assembled onto one TMA. Briefly, 1-2 tissue cores at diameters of 0.6 and 1.5 mm were obtained from areas containing high-density tumor and for TMA construction and RNA extraction as described previously (Rickman et al., 2010). All patients' information and samples were collected with approval from the local institutional review board at the Universiy of Calgary, Calgary, Alberta.

Assessment of ERG gene rearrangement by Fluorescent In Situ Hybridization and immunohistochemistry (IHC). ERG gene rearrangement status was evaluated using a break-apart probe assay and IHC on a TMAs of 52 patients (54 samples) with CRPC treated by different androgen deprivation therapies as previously described (Bismar et al., 2011).

Expression Profiling. The inventor reanalyzed gene expression data, which was previously generated using a complementary DNA-mediated annealing selection and ligation assay (DASL; Illumina, San Diego, Calif.) to profile the tumor samples from formalin-fixed paraffin embedded (FFPE) tissue (Rickman et al., 2010). Full disribtion of the DASL methods are provided in supplementary methods Immunohistochemistry (IHC)

The inventor used the Vantana autostainer system (NEXES IHC model) to assess the protein expression by IHC. Primary antibodies used and their conditions are listed in Table 1. Briefly, 4 μm thick sections from formalin-fixed paraffin-embedded tissue blocks were cut and placed on plus charged slides. Prior to the staining, heat induced antigen retrieval was carried out by vegetable steamer in sodium citrate (pH 6.0) or EDTA (pH 9.0) antigen retrieval buffer for 10 or 20 minutes, and then cooling down to room temperature for about 20 min. The slides were incubated for 60 minutes at 37° C. with primary antibodies at corresponding dilution (Table 1). A Ventana iView DAB detection kit (Ventana Tucson, Ariz., USA) was used for HRP detection following by hematoxylin counter stain. A multi-tissue control TMA slide containing samples of prostate, colon and breast was used as positive and negative control. Negative controls were performed by omitting the primary antibody and substituting it with normal mouse 1/200 prediluted serum (Ventana, Ark., USA).

DNA, RNA, and Protein Purification. DNA, RNA, and protein were extracted from fresh-frozen samples of paired benign prostate and localized prostate cancer tissue (n=10), or lymph node (LN) metastasis (n=3) tissue surgically extracted from patients, using the TriplePrep Isolation Kit (GE Healthcare) according to manufacturer's protocol (protein samples were resuspended in 7 M urea, rather than the recommended 2×DIGE buffer, for subsequent SDS-PAGE and western blot analysis). Total protein was extracted from prostate cancer cell lines by sonication in RIPA Lysis Buffer (Sigma) supplemented with cOmplete EDTA-Free protease inhibitor cocktail (Roche Applied Sciences) according to manufacturer's protocol. For prostate cancer cell lines, total RNA was isolated using Trizol (Invitrogen, Carlsbad, Calif.) according to manufacturer's protocol. RNA Samples were quantified using a NanoDrop 2000 spectrophotometer (Thermo Scientific, Wilmington, Del.) according to provided instructions, and A260/A280 ratios were 1.8-2.1 for all RNA samples. Protein samples were quantified by Bradford Assay (Bio-Rad) using a PowerWave XS plate reader (BioTek, Winooki, Vt.).

Quantitative RT-PCR Analysis. For each RNA sample, 300 ng of total RNA was used as template in the reverse transcription reaction using qScript™ cDNA SuperMix (Quanta BioSciences, Gaithersburg, Md.) in a 20 μl volume according to manufacturer's protocol. Real-time PCR was performed on the ABI StepOne Plus system (Applied Biosystems, Carlsbad, Calif.) using PerfeCTa™ SYBR Green FastMix™ (Quanta BioSciences, Gaithersburg, Md.). Briefly, 1 μl of cDNA product was used as template in a 20 μl PCR reaction containing 10 μl of SYBR Green FastMix (2×), 0.5 μl of each gene-specific primer (10 μM) and 8 μl of nuclease-free water. The following amplification protocol was performed: 95° C. for 2 min, followed by 40 cycles of 95° C./10 s, 60° C./30 s. Fluorescence signals were detected during the 60° C./30 s step. Baseline and threshold cycle number (Ct) were determined automatically. Water was used as a no-template control for the PCR reaction. Beta-glucoronidase (GUSB) and glyceraldehyde 3-phosphate dehydrogenase (GAPDH) were selected as the most stable reference genes, and relative expression of target genes in localized prostate cancer was calculated using the comparative (ΔΔCt) method with the paired benign tissues as the reference samples. As paired control samples for the LN metastasis tissue were not available, relative expression in these samples was calculated using the average Ct value for the benign prostate tissue. Analysis of relative target gene expression in ERG negative and ERG positive tumours was performed by pooling ΔCt values of those samples and using the mean ΔCt of ERG negative as the reference sample.

Pathological analysis. All TMA cores were reviewed and assigned a diagnosis (i.e., benign or prostate cancer) by the study pathologists (LHT and TAB). Protein intensity expression was assessed semi-quantitatively using a four-tiered system (0, negative; 1, weak; 2, moderate; and 3, strong) without prior knowledge of clinical information.

Statistical analysis. One-way ANOVA tests were performed in order to determine if the scores for each marker were significantly different between benign (BN), localized (PCA), and castration-resistant (CRPCA) prostate cancer. Data for each of the 10 markers from the 4-tiered immunohistochemistry analyses were used for these tests. Phi correlations were used to test each marker's association with ERG status in prostate cancer samples. The data from the ERG immunohistochemistry was grouped where a score of 0 was considered no expression (corresponding to no rearrangents) t and scores of 1, 2, or 3 (weak, moderate or high ERG expression) were counted as reflective of ERG rearrangement (based on previous FISH and IHC correlative data; not shown) having taken place. The IHC results for the markers were grouped into two categories (negative and weak) versus (modertae and high) expression. Analyses were performed using SPSS v.16 (IBM Corporation, Somers, N.Y.). A two-tailed p-value ≦0.05 was considered statistically significant. Statistical significance in the real-time PCR data was determined with Student's t-test and differences were considered significant only when the P-value was <0.05.

Expression Profiling. Briefly, the DASL method exponentially amplifies transcripts of interest and has high sensitivity for genes with low expression levels. The platform used previously consisted of four DASL assay panels (DAP) covering approximately 6000 genes selected based on the evaluation of more than 250 microarray data sets (database available at world-wide web at broad.mit.edu/cancer/pub/HCC).

The samples were originally jointly analyzed with another cohort representing hormone naive tumors in the Swedish Watchful Waiting cohort. To resolve the potential study design confounder (expression data for the two cohorts were generated at different time in different centers), gene expression data from nine clinically localized prostate cancer (experiment control samples), which were profiled together with this cohort in the same experiment. Data were subjected to quality assessment by completion of morphologic evaluation and gene expression profiling. Interpatient and intrapatient sample correlations were assessed. In this process, when more than one tumor focus was present for a given patient from a single or multiple biopsies, we retained data only from foci with intrapatient differences in gene expression (correlation coefficient <0.7). Data were available from fifty-four tumor samples belonging to 52 individuals that passed these quality control measures.

Cell culture and reagents. The prostate cancer cell line PC-3 was modified to overexpress TMPRSS2-ERG or control luciferase vector were obtained from Dr. Felix Feng (University of Michigan, Ann Arbor, Mich.). TMPRSS2 (Exon 1)-ERG_variant1 (Exon 2) was PCR amplified from VCaP cell line cDNA and cloned into the pLenti6.0 vector system (Invitrogen, Carlsbad, Calif.). Luciferase cDNA was obtained from the University of Michigan vector core and cloned into the system as a control. Lentivirus was created according to standard protocol. Briefly, HEK293 cells were co-transfected with the lentiviral construct and packaging plasmids using Fugene 6.0 according to the manufacturer's protocol (Roche). Viral supernatant was collected 48, 60 and 72 hours after transfection, filtered and used to transduce PC3 cells in the presence of 4 μg/mL polybrene (Sigma). Seventy-two hours later, cells were selected in 3 μg/mL blastacidin until selection was complete. ERG overexpression was monitored by Western blot analysis. Genetic identity of the cell line was confirmed by genotyping using the Profiler Plus system available from Promega. Cells were grown in RPMI 1640 medium (Invitrogen, Carlsbad, Calif.) supplemented with 10% fetal bovine serum (FBS) and were maintained at 37° C. in 5% CO₂.

Western blot analysis. Proteins from total cell (20 μg) or prostate tissue (benign, cancer, or LN mets; 10 μg) lysates were separated by SDS-PAGE and transferred to polyvinylidine fluoride (PVDF) membranes. Primary antibodies used are described in Table 2, except for ING3 (Epitomics 3213-1 rabbit monoclonal antibody selected for western blot analysis). GAPDH (2118, Cell Signaling Technology, Danvers, Mass.) was used as a loading control. HRP-conjugated anti-rabbit and anti-mouse secondary antibodies (7074 and 7076, respectively, from Cell Signaling Technology, Danvers, Mass.) were used for detection with Amersham ECL Prime (GE Healthcare, Piscataway, N.J.) and exposed to Amersham Hyperfilm ECL (GE Healthcare).

### Example 2

### Results

Computational Analysis and Characterization of Significant Differentially Expressed Genes. The inventor used the Singular Value Decomposition (SVD) (Varshaysky and Gottlieb, 2006) based gene selection method to discover highly differentially expressed genes ranked based on their contribution to the overall entropy of the expression data. Genes that were highly ranked were identified as potential biomarkers discriminating between ERG rearranged (positive) and ERG non-rearranged (negative) samples. Forty-six patient samples with known ERG status, based on fluorescence in situ hybridization (FISH) and IHC ERG expression, were used in this study; 27 fusion-negative (ERG0) samples and 19 fusion-positive (ERG1) samples were identifed.

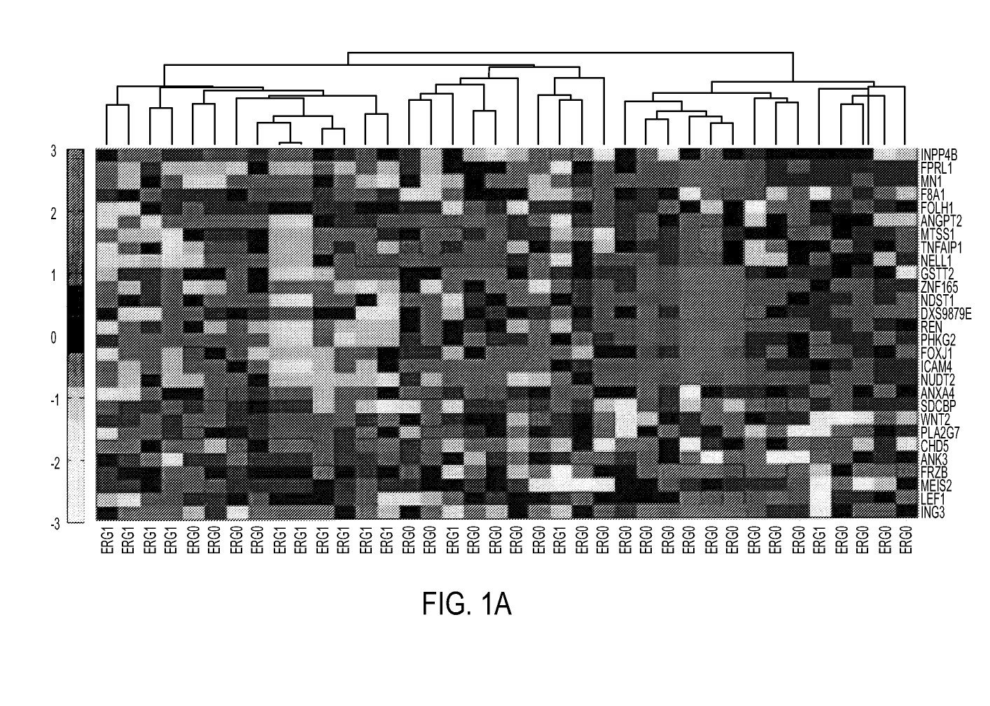

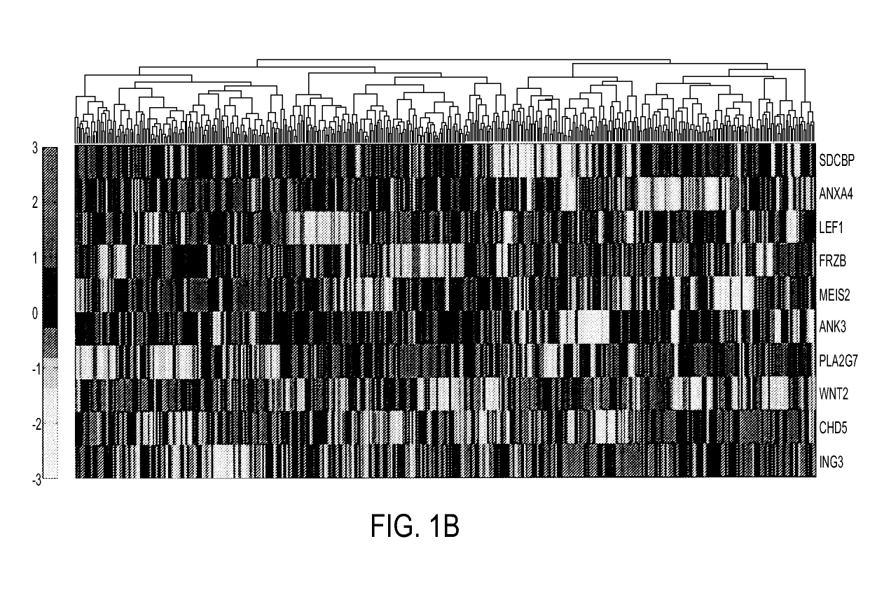

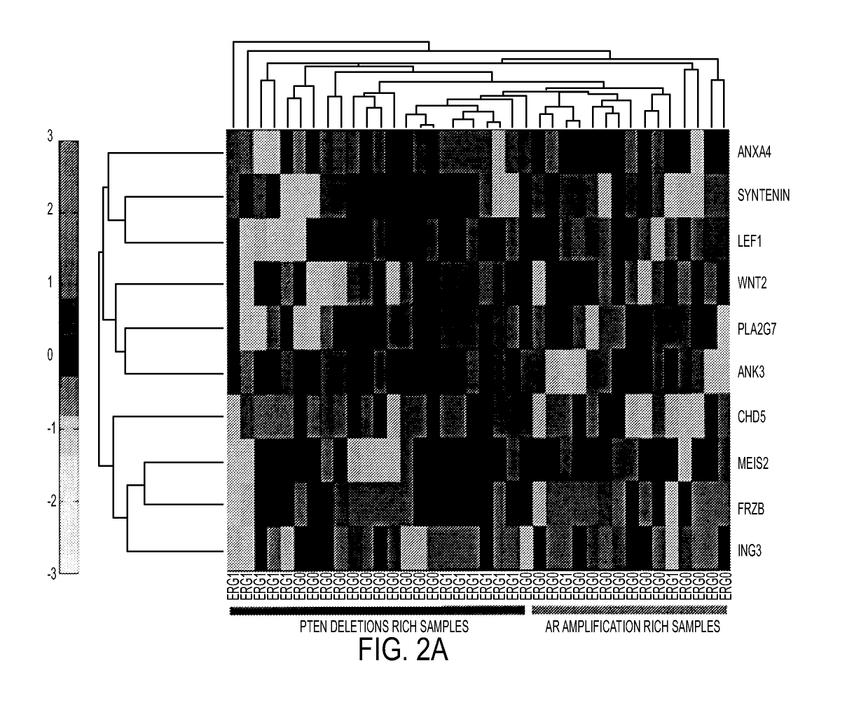

Based on SVD analysis, a list of 28 candidate genes were identified that showed significant changes in expression between ERG0 and ERG1 samples (FIG. 1A). Genes that have SVD value larger than (Average+2*Standard deviation) are selected as significant. The 28 genes list was able to classifiy ERG samples with 70% accuracy using Support Vector Machine (SVM). Next, the inventor narrowed down the 28 genes by removing genes that do not influnce the classification accuracy by removing one gene at a time. If a gene removal improved the accuracy, then it was included; however, if it decreased the accuracy then it was removed. The final list included a 10 genes list which was able to accuratly predict ERG status for a given sample with 76% accuracy using linear SVM (FIG. 1B). To validate this 10-gene signature further, the inventor applied it to an independent public data set (Swedish cohort GSE8402). The 10-gene signature was able to achieve 83% accuracy rate in predicting the ERG status of each sample in the Swedish cohort (SFIG. 1A). The inventor also investigated the power of this 10-gene list on two additional publically published prostate cancer cohorts. As the ERG gene rearrengemnets status were unknown for those cohorts, the inventor grouped the samples (ERG 1 versus ERG 0) based on the expression level of ERG mRNA which was previously shown to be reflective of ERG gene rearrangments status (Bismar et al., 2011). The 10-gene list was able to accurately predict ERG status in 82% and 65% of samples in the Tylor and Glinsky data, respectively (Taylor et al., 2010; Glinsky et al., 2004) (data not shown).

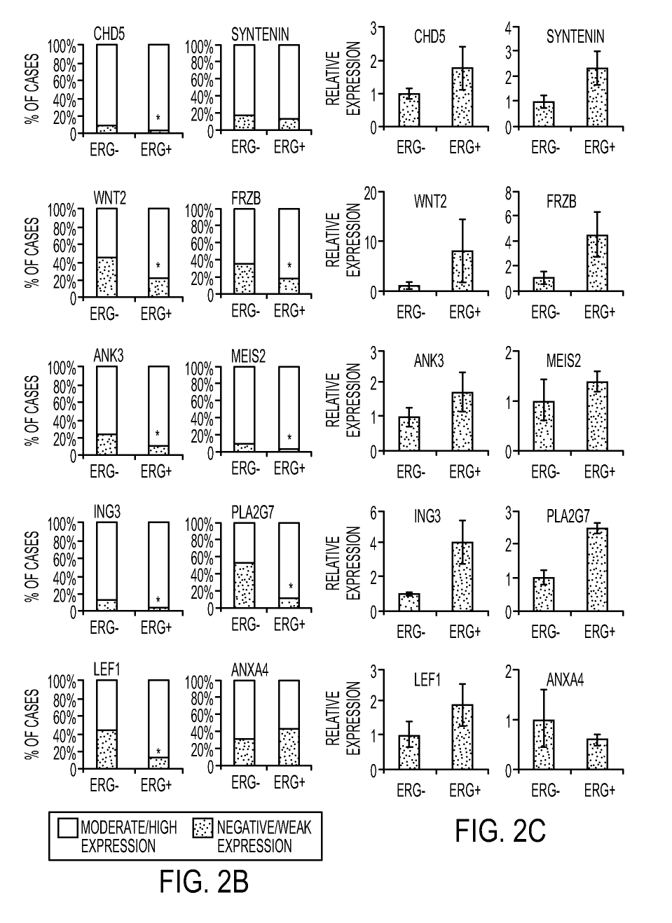

As previous reports had suggested significant association between ERG gene rearrangements and PTEN deletions with potential implication of the Androgen receptor, the inventor sought to investigate whether PTEN and AR status show any clustering within the gene list. The inventor observed two clusters of tumors within the 46 samples. The first was enriched for PTEN deletions and the second was enriched for AR amplification (FIG. 2A). This may explain in part, the inability of the 10 gene model to 100% accuratey predict ERG status.

Based on the above observation, the inventor next grouped the samples based on both ERG/PTEN status and ERG/AR status. The ability for 10-gene signature to accurately predict a sample status improved to ˜82% when both ERG/PTEN or ERG/AR status was concidered. This was even more robust in the Tylor data reaching ˜92% accuracy (data not shown). This highlights the heterogenous nature of prostate cancer and further confimr significant interlation between ERG, PTEN and AR

This promted the inventor to investigate individual gene expression/-fold change for individual genes in the 10-gene signature relative to ERG and ERG/PTEN. The inventor observed that the majority of those genes kept the same expression direction and had similar-fold change when PTEN status was considered, with the exception of MEIS2 and Syntenin which changed direction when PTEN status was inlcuded. suggesting an association between MEIS2 and Syntenin with PTEN (FIG. 1B).

Association of mRNA and Protien Levels of Identified Genes with ERG mRNA and Protien Expression. To validate the association of the genes identified by bioinformatic analysis with ERG gene rearrangement, the inventor analyzed the protien expression of those genes in comparion to ERG protien expression using a progression tissue microarray (TMA) of 86 patinets (320 cores). He performed IHC analysis using antibodies to the 10 markers. In each subset of patient samples (i.e., ERG0 or ERG1), the intensity level for each marker was categorized as absent/weak versus moderate/strong. Eight of 10 markers (CHD5, Ankyrin, MEIS2, FRZB, LEF1, PLA2G7, ANXA4 and WNT2) showed significant changes in the expression levels relative to ERG protein (FIG. 2B). Significant association of syntenin and ING3 protien levels to ERG protien were conformed on another larger cohort of localized PCA (data not shown). Western blot of all ten markers was also confirmed by assessing protein isolated from the same samples used above relative to ERG protien expression (data not shown).

To confirm the association between identifed markers and ERG at the mRNA level, the inventor assessed the relative expression of ERG in a small cohort of frozen patient samples (benign prostate, localized prostate cancer, and metastases) by qRT-PCR using primers specific for all known TMPRSS2-ERG fusion variants. In all cases where ERG was expressed highly relative to matched benign samples, TMPRSS2-ERG transcripts were detected (data not shown). The samples were categorized into ERG-negative and ERG-positive groups, and the mean relative expression of these samples was calculated. Similar to the results obtained on the protien level, mRNA relative expression levels of the 10 genes were differentially expressed relative to mRNA ERG mRNA levels being higher in ERG positive samples, except for ANXA4, where it was lower, consistent with IHC data (FIG. 1C). However, the changes in relative expression were only shown to be significant in PLA2G7 (p<0.05), possibly reflecting small sample sizes in each category. To confirm the associations of IHC and mRNA observed in clinical samples, the inventor performed western blot and qRT-PCR analysis on the prostate cancer cell line, PC-3, that had been stably transfected with a TMPRSS2-ERG or luciferase control expression vector (data not shown). However, some of results were not consistent with those observed in the clinical samples, suggesting that heterogeneity or cell line charesteristics may have additional influence on the relative expression levels for each gene. Nevertheless, these data validate the genes identified by SVD analysis to be related to ERG both in clinical samples as well as in vitro ERG model.

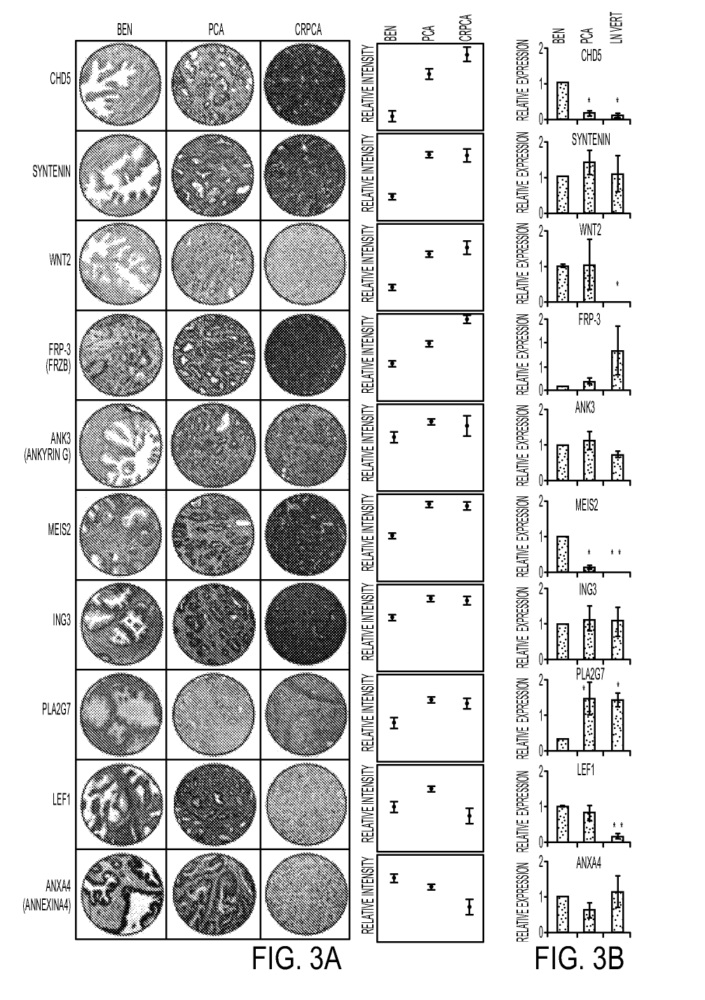

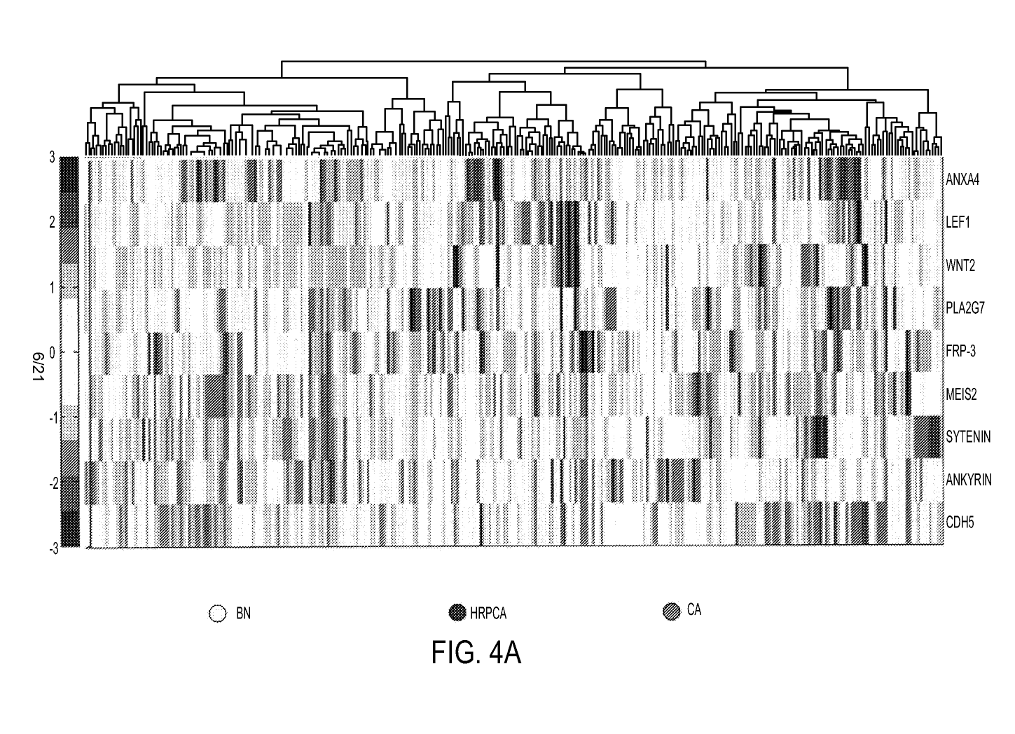

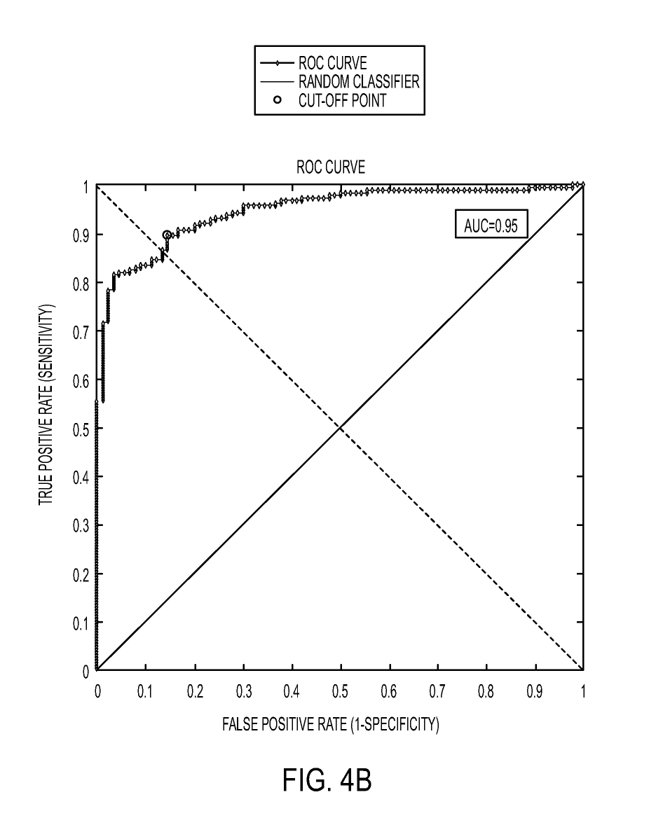

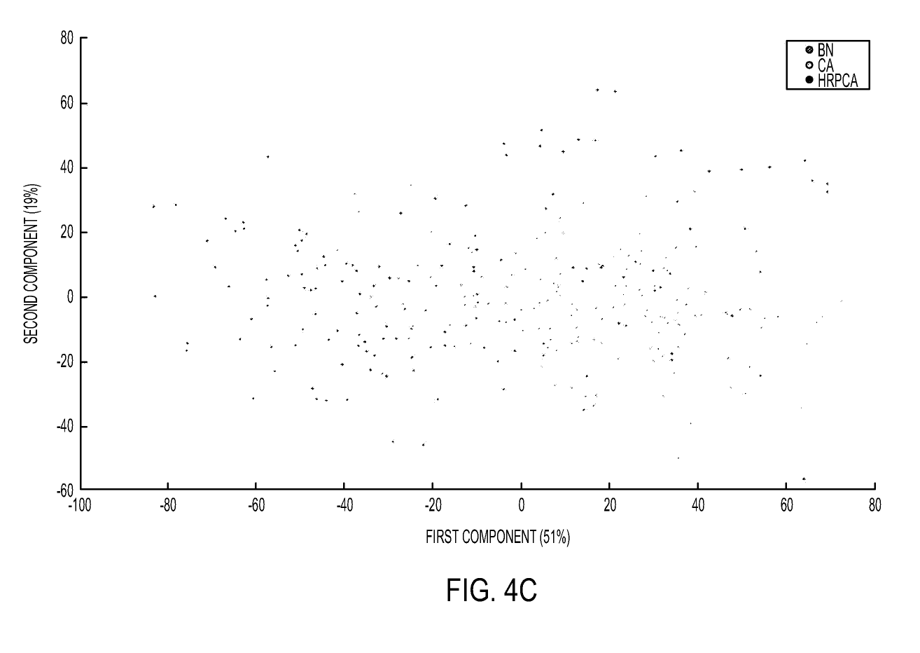

Association of Protien Expression Levels of Identified genes with Prostate Cancer Progression. To assess the significance of the 10-gene signature in disease progression regardless of ERG status, the inventor analyzed the level of expression leveles of the 10 markers relative to benign, localized and castration resistant prostate cancer using the progression TMA described above. Each marker was plotted relative to disease progression. The protein levels of each of the ten markers were significantly associated with prostate cancer cancer progression. (FIG. 3A; p<0.001). All markers except ANXA4 showed significant overexpression whereas ANXA4 was significantly down-regulated with prostate cancer progression, consistent with earlier IHC data relative to ERG protien expression.

The inventor also verified the relative mRNA expression level of the 10 genes in limited patients' samples (benign, localized and metastatic PCA) by quantitative RT-PCR (FIG. 3B). The expression levels of most markers in the frozen samples were concordant with the IHC data for tumor progression, with a few notable exceptions: CHD5 and MEIS2 were significantly downregulated in PCA and metastatic tissues, as compared to benign tissue (FIG. 3B; p<0.05), and WNT2 was significantly lower in LN Mets samples (p<0.05); whereas the protein levels for those markers are significantly higher in similar tissues by IHC analysis (FIG. 3A).

ERG Gene Signature In Relation Prostate Cancer Patients' Prognosis and Overall Survival. To investigate association to patients' prognosis, the inventor analyzed the 10-gene signature using publically available patients' datasets. As the signature is reflective of ERG expression, the inventor decided to investigate how the gene sigature compare to ERG expression alone in predicting disease-specific survival in several prostate cohorts. The inventor investigated the association of ERG status to patients' prognosis in three prostate datasets (Tylor, Glinsky and the Swedish cohorts). For the Swedish cohort, ERG gene arrangemnets status was determined by FISH. However, in the case of Tylor and Glinsky datasets, the inventor predicted the ERG status based on ERG gene expression (as FISH data was not available and earlier data confirmed significant asociation between ERG gene rearrangments and ERG mRNA levels).

Samples that had ERG expression above the interquartile range (IQR) were considered ERG positive samples. Based on ERG status, the inventor grouped samples into ERG 1 (fusion-positive) and ERG 0 (fusion-negative). He also used the 10-gene model to group patients using pearson correlation into ERG1-like and ERG0-like patients based on the expression level of the 10-genes. ERG1-like samples are those that are overexpressed in (CHD5, WNT2, PLA2G7, ANK3, SDCBP) and under expressed in (ING3, MEIS2, FRZB, LEF1, ANXA4), and ERG0-like samples have the opposite expression profile to ERG1-likesamples. As PCA is known to be of significant hetergeneity, the inventor hypothesized that ERG status alone might not be very predictive of patients' prognosis, and that integrating other gene's status might improve overall patients' prognosis.

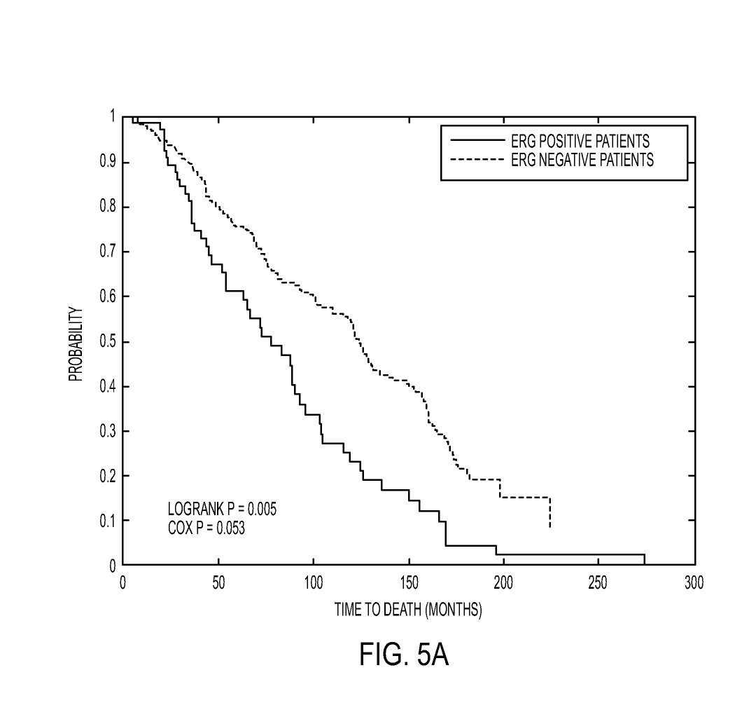

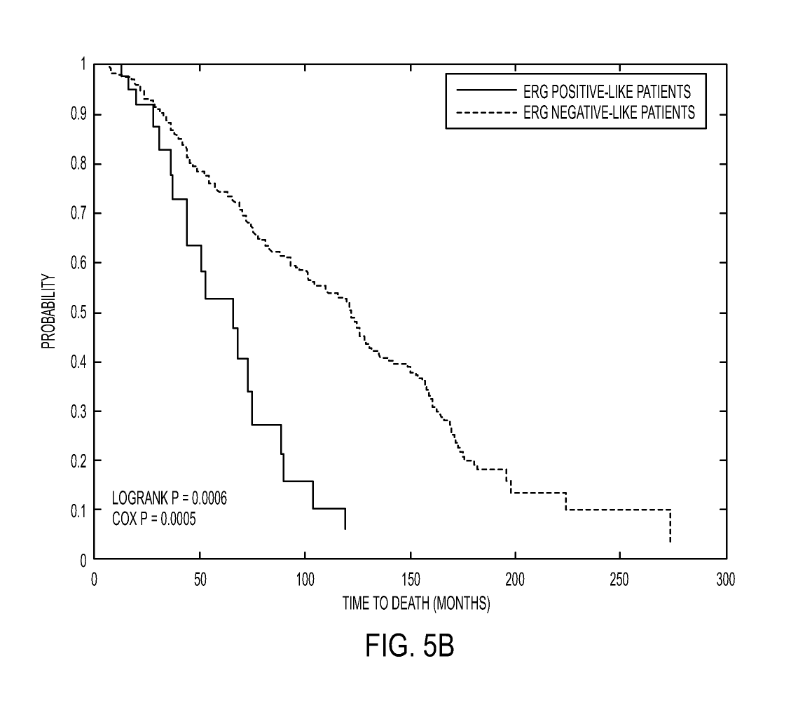

In the Swedish cohort patients were stratified into high and low risk groups based on ERG status with. Patients harboring ERG gene rearrangements (n=64) were at higher risk compared to patients with no ERG gene rearrangements (n=217) (p=0.005 logrank test; HR:1.25) (FIG. 5A). When the inventor implemented the 10-gene signature onto this cohort to predict patients' prognosis, the 10-gene model separated patients into ERG1-like (n=22; 7 of them were ERG1) and ERG0-like patients (n=259). This model showed stronger association to patients' prgnosis compared to ERG status alone (p=0.00062 logrank test; HR:2.38) (FIG. 5B). Multivariate analysis using cox proportional hazard mode, confirmed that the 10-gene model to be more significantly associated with overall survival compared with the ERG status alone (p=0.0032 versus p=0.053). Multivariate analysis also demonstrated that PLA2G7 and ANXA4 are highly associated with the outcome (p=0.00007 and p=0.02), respectively.

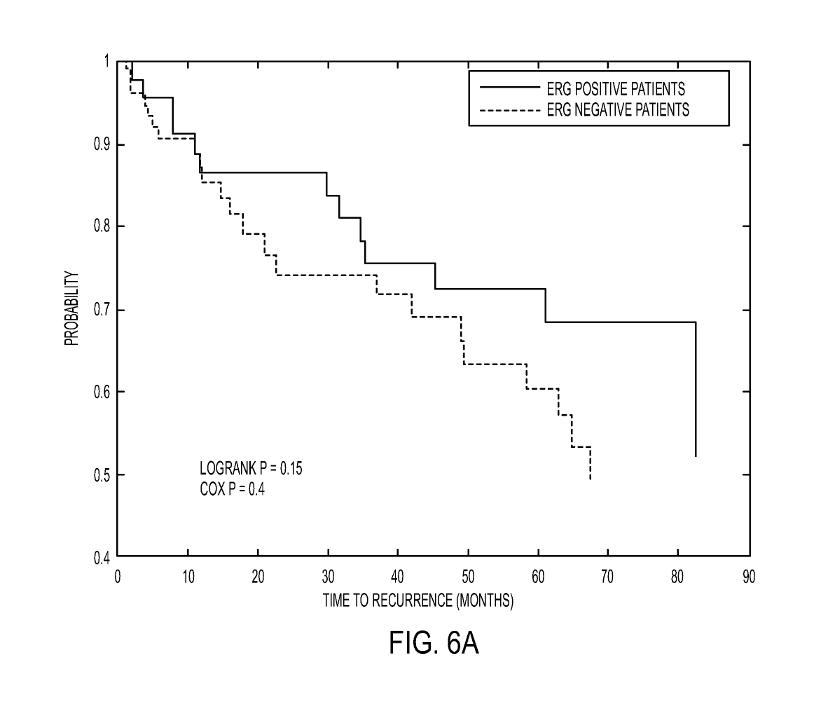

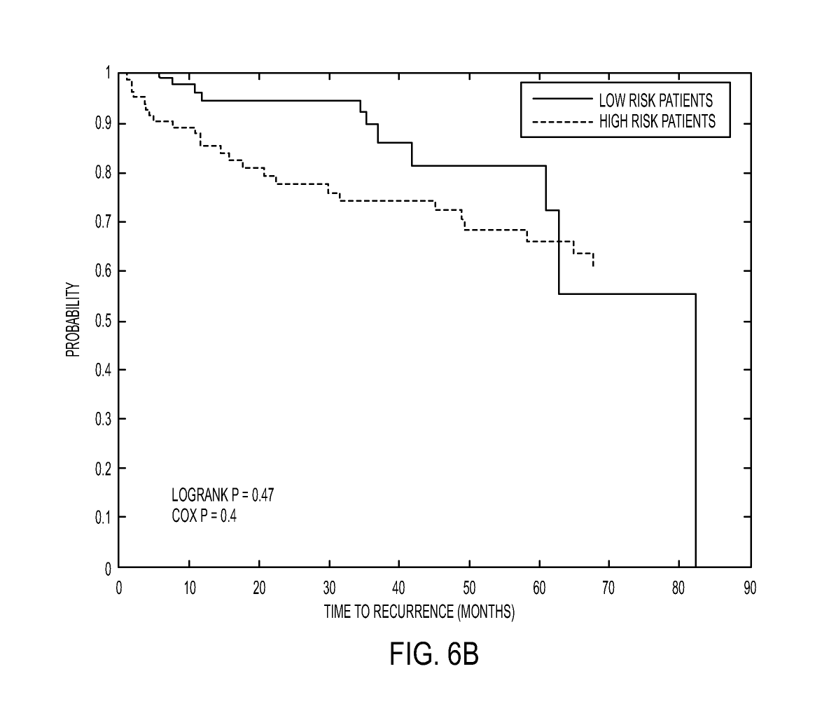

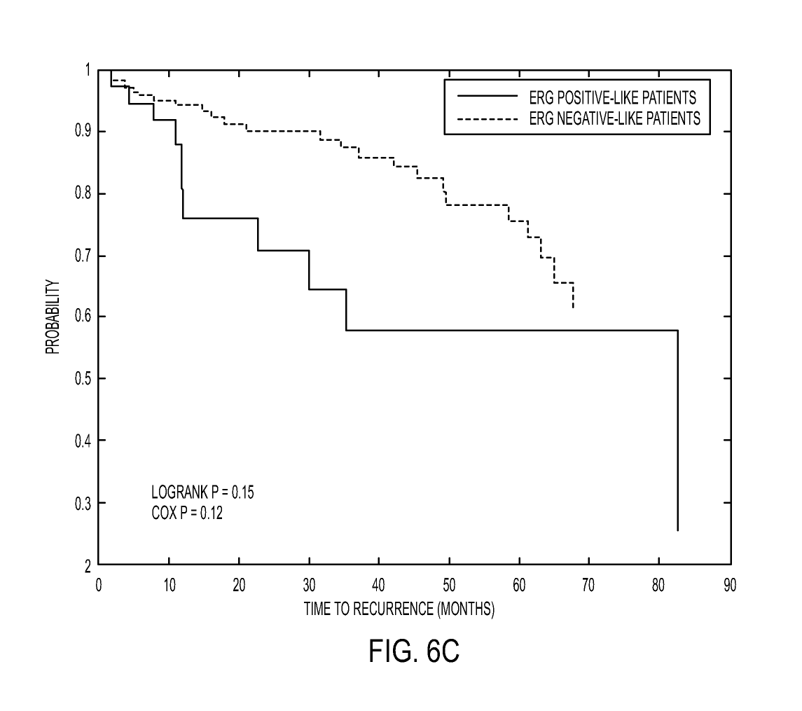

To further validate the significance of the 10 gene signature (ERG1-like) to predict aggressive prostate cancer, the inventor applied this model on Gilinsky and Tylor data. First, he grouped patients in Glinsky data based on ERG expression level. Patients with high ERG (n=32) were not well separated from patients with low ERG (n=47) (p=0.15, HR:0.76) (FIG. 6A). He further grouped samples into high risk (n=21) and low risk patients (n=58) based on ERG status as described in Varambally et al. (2005). The results still showed poor separation (p=0.47, HR:1.4) (FIG. 6B). Based on signature of the 10-gene model, the inventor grouped samples into ERG1-like (n=18, 11 of them are ERG1) and ERG0-like (n=61). This 10-gene signature was able to significantly separate patients with ERG1-like profile from ERG0-like patients (p=0.15, HR:1.8) (FIG. 6C). Using multivariate analysis, the 10-gene model was more associated with prognosis and death (p=0.1) compared to ERG alone (p=0.41).

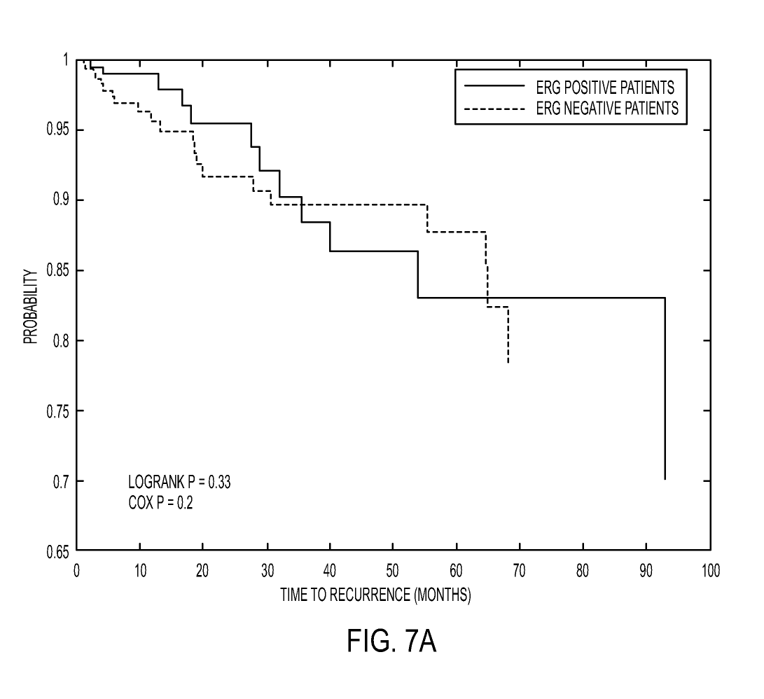

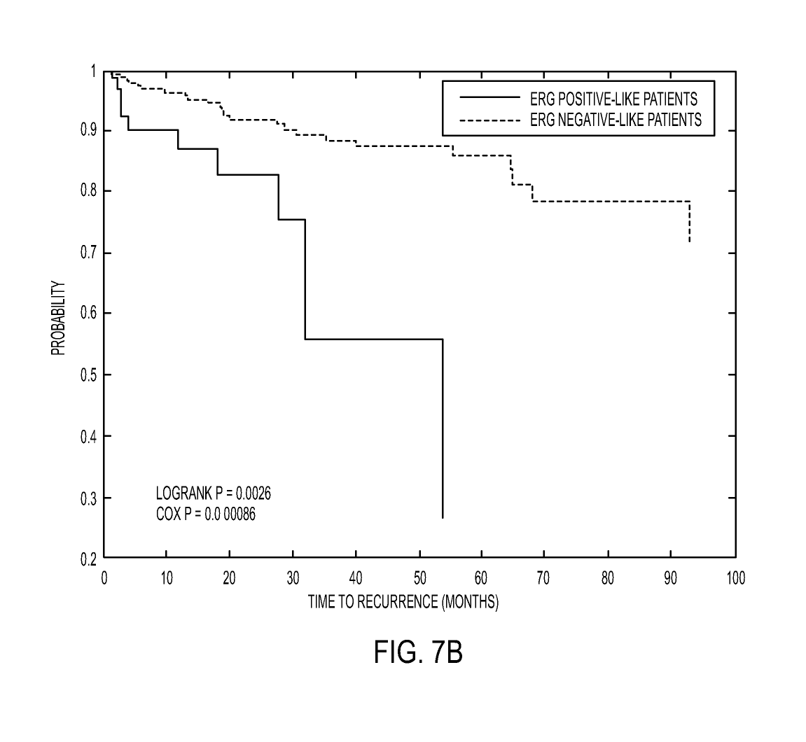

The inventor also assessed the prognostic significance of ERG expression and the 10-gene model on cancer recurrence and cancer aggressivnes using the Tylor data. ERG expression was not efficient to separate patients into clinically distict groups (p=0.33, HR:0.7) (FIG. 7A). However, the 10-gene model successfully separated patients in high risk group (ERG1-like) (n=23; 9 of them are ERG1) versus low risk ERG0-like group (n=117) (P=0.0026, HR:3.2) (FIG. 7B).

Finally, the inventor tested whether 10-gene model is able to classify samples with very aggressive form of PCA (high Gleason score and high metastatic; i.e., cluster 5,6 in Tylor data) from the other clusters. SVM based classification domenstared that the 10-gene model predicts the clinical cluster of patients slightly more accurate than ERG status alone (80%; HR:1.07 versus 77%; HR: 0.87). Multivariate analysis also showed that MEIS2 (p=0.00001) and FRZB (0.0048) are significantly associated with cancer recurrence. These data confirm that the expression signature of the 10-gene model is more directly able to starify patients into low and high rsik groups than is the ERG status alone.

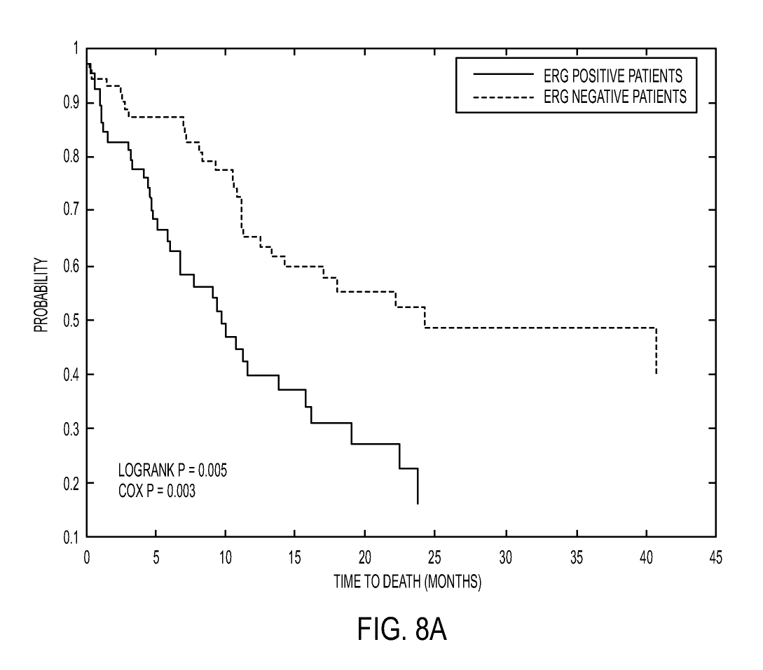

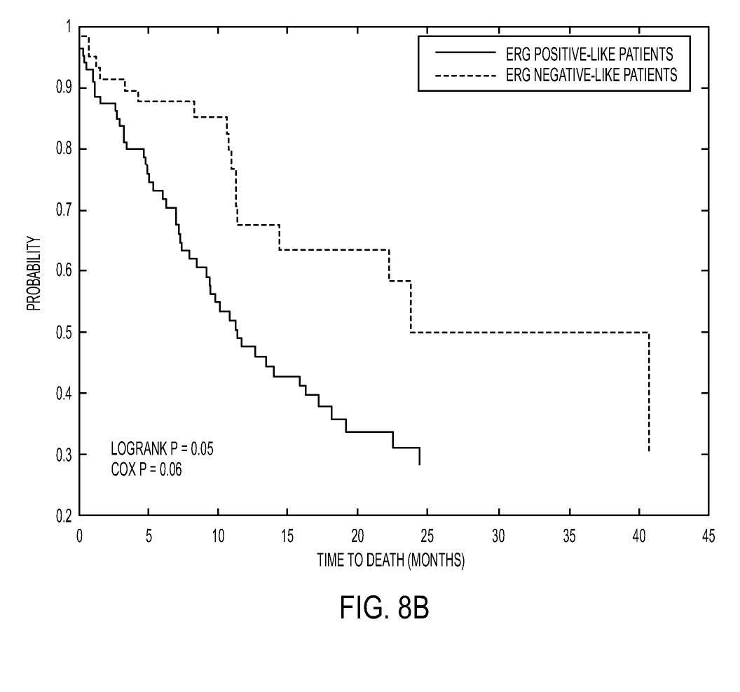

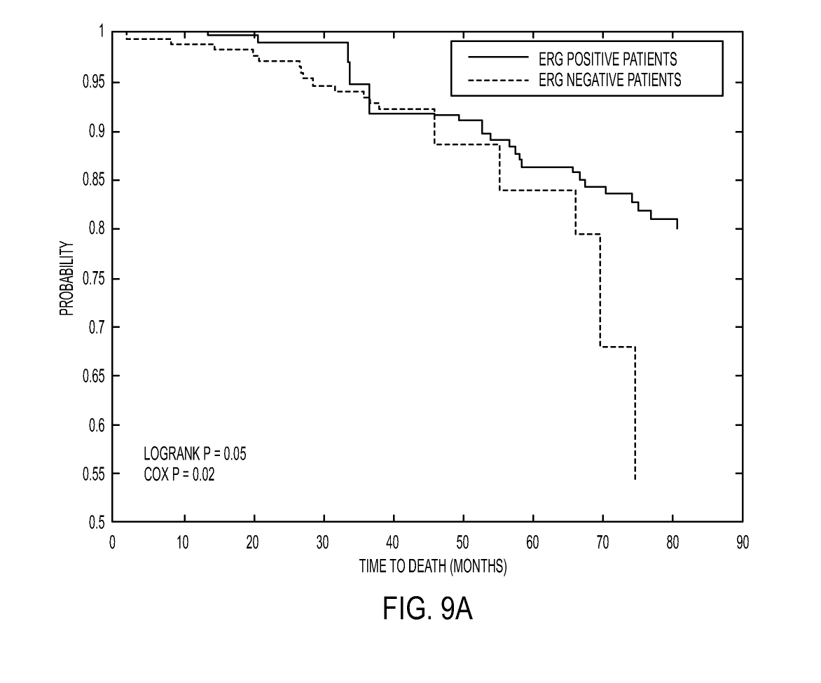

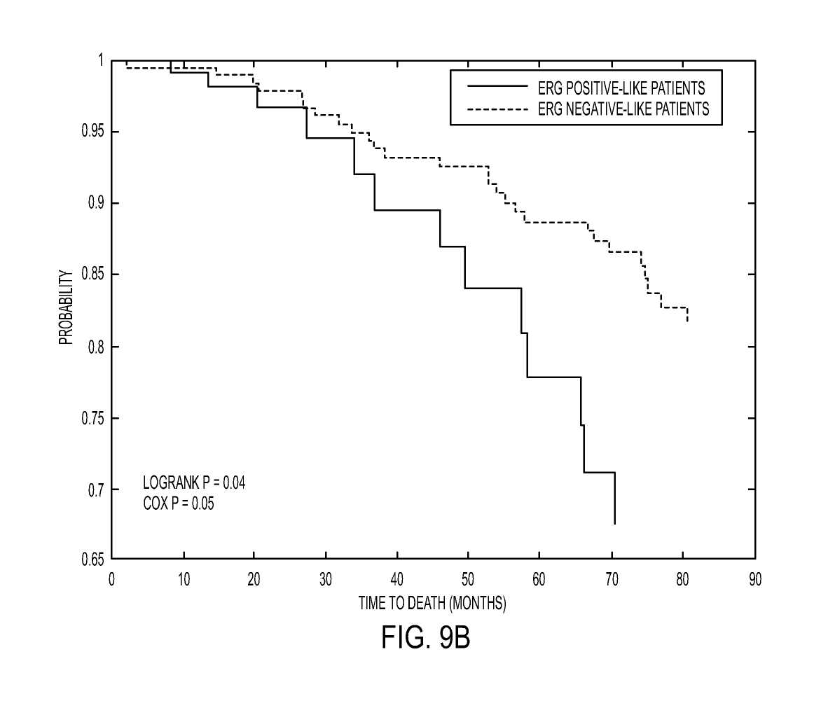

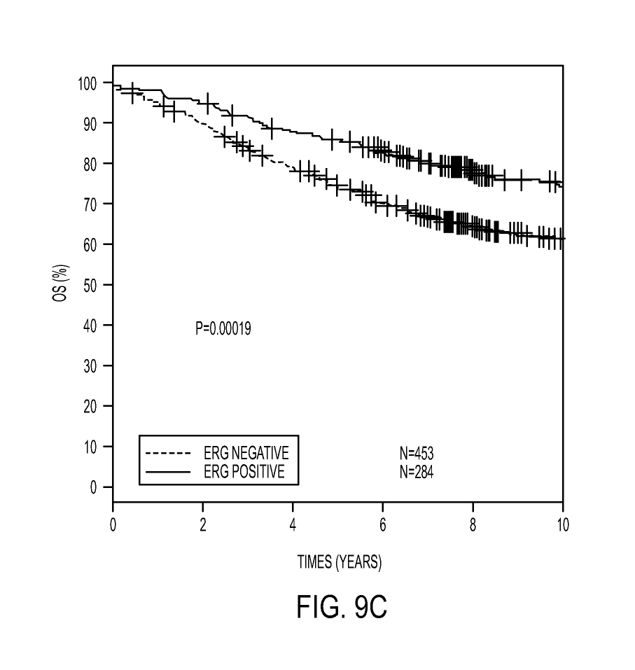

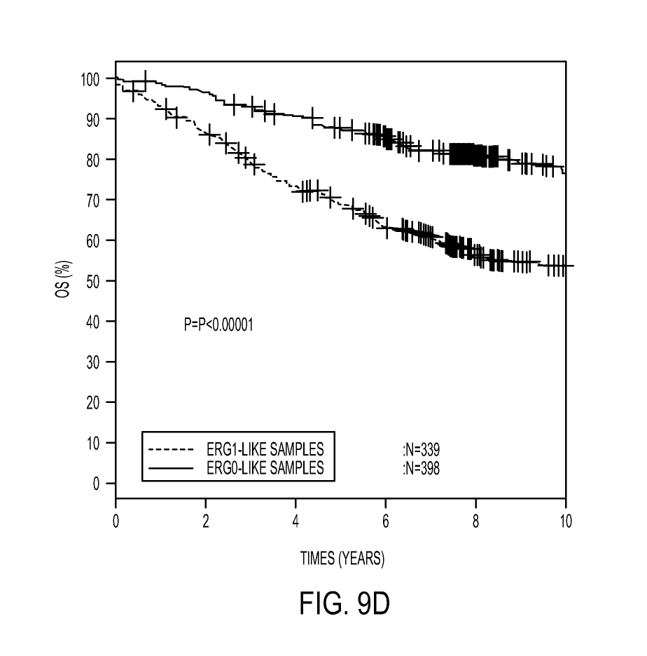

ERG Gene Sigature Relative to Patients' Overall Survival in Other Maligancies. As this 10-gene signature was related to ERG and associate with aggressive phnotypes of prostate cancer, the inventor next asked whether the it can predict prognosis and survival in other malinacies which also express ERG. To assess the prognostic capability of the 10-gene model, he used a published leukemia datset (Bullinger et al., 2004) and a Swedish breast cancer data set (GSE1456) (Pawitan et al., 2005). The inventor grouped leukemia samples based on their ERG expression level based on IQR. Using ERG status as a prognostic biomarker, samples were separated into ERG1 patients (n=56) and ERG0 (n=60) (logrank test, p=0.005; HR:2.12) (FIG. 8A). Using the 10-gene model as prognostic biomarker, results revealed that patients are still well separated into ERG1-like (n=78) and ERG0-like (n=38) (logrank test, p=0.02, HR:1.4) (FIG. 8B). Multivariate analysis also confirmed significant association between both ERG status and the 10-gene model with over all survival (p=0.003) and (p=0.01), respectively. Multivariate analysis using cox regression analysis also showed that PLA2G7 and LEF1 were significantly associated with overall survival (p=0.006) and (p=0.04), respectively. This further demonstrates that both ERG status and the ERG-like 10-gene model are clinically prognostic for patients' survival in leukemia, and that ERG1 and ERG1-like patients are at higher risk as in PCA patients. The inventor then tested the Swedish breast cancer data (GSE1456) to assess the prognositc capability of ERG and ERG-like siganture. ERG status demonstarated a good clinical biomarker to separate ERG1 (n=136) from ERG0 patients (n=23) (logrank test, p=0.06; HR:0.52). However, in this analysis, ERG1 patients were associated with better overall survival than ERG0 patients, contrary what to what would have been expected. (FIG. 9A). Using the 10-gene model to separate patients into ERG1-like (n=34) and ERG0-like (n=125), the inventor was able to separate patients into two prognostic groups (p=0.05; HR:2). However, in concordance to previous results, patients with ERG1-like signature were associated with worse prognosis compared to ERG0-like signature (FIG. 9B). This is in line with the inventor's hypothesis that the ERG1-like 10-gene signature is more accurate than ERG alone in stratifying patients into high and low risk groups and is associated with agrressive cancer phenotypes. To further test the association of ERG status with breast progression, the inventor tested it on a larger cohort of patients using GOBO (Ringner et al., 2011), an online tool that assesses the association of genes with outcome in more than one thousand breast cancer patients' samples data set. First, the inventor was interested in investigating whether ERG expression is associated with patient's prognosis in breast cancer. This analysis using GOBO confirmed previous results that tumors with low ERG levels (ERG-negative) had favorable prognosis and were significantly separated from high ERG level (ERG-positive) tumors which showed worse prognosis (log-rank p=0.00019; HR:1.35) (FIG. 9C). As the inventor could not employ the 10-gene signature in the GOBO databases (as there were over- and under-expressed tumors), the inventor sought to investigate the significance of the 4 genes (ING3, FRZB, LEF1, ANXA4) under-expressed in the ERG1-like signature (based on the 10-gene model) as prognostic biomarkers. The four genes were significantly associated with worse patients' prognosis (log-rank p<0.000001, HR:1.8) (FIG. 9D). Using the other 6 genes overexpressed in ERG1-like siganture, the inventor was unable to demonstrate any prognostic significance with overall survival (p=0.9). Using the 10-gene model together still showed good performance as prognostic biomarkers (p=0.00017). However, this may be due to the effect of the 4-gene signature, as the inventor was not able to employ the complete 10-gene signature as reflective of ERG using the GOBO dataset (i.e., same direction of over- and under-expressed genes discovered). The inventor further investigated the 4-gene siganture in ER-positive and HER2-enriched tumors. The 4-gene signature was able to separate the ER-positive tumors and HER2-enriched samples into high and low risk groups (p<0.00001) and (p=0.0034), respectively. This might indicate that the 4-gene signature also plays a significant role in stratifying patinets within the “good group” and the “bad group” representing ER-positive and HER2-enriched tumors.

TABLE 1
Fold change of the 10-gene set signature that showed to have significant
impact on the entropy of the gene expression data.

| | | ERG1 | ERG1_PTEN3 |
|---|---|---|---|
| | | to | to |
| Gene | GenBank ID | ERG0 | ERG0_PTEN0 |
| Inhibitor of growth family, | NM_019071.2 | 0.45* | 0.45* |
| member 3 (ING3) | | | |
| Lymphoid enhancer-binding | NM_016269.4 | 0.82 | 0.73 |
| factor 1 (LEF1) | | | |
| Meis homeobox 2 (MEIS2) | NM_170677.3 | 1.12 | 0.47* |
| frizzled-related protein | NM_001463.3 | 0.58* | 0.52* |
| (FRZB) | | | |
| Ankyrin 3, node of Ranvier | NM_020987.3 | 1.24 | 1.34 |
| (ANK3) | | | |
| Chromodomain helicase DNA | NM_015557.2 | 1.70* | 1.88 |
| binding protein 5 (CHD5) | | | |
| Phospholipase A2, group VII | NM_005084.3 | 1.40* | 1.53 |
| (PLA2G7) | | | |
| wingless-type MMTV | NM_003391.2 | 1.36* | 1.61* |
| integration site family | | | |
| member 2 (WNT2) | | | |
| Syndecan binding protein | NM_005625.3 | 0.97 | 1.09 |
| (Syntenin) | | | |
| Annexin A4 (ANXA4) | NM_001153.3 | 0.57* | 0.60* |

Some genes (Syntenin) do not show significant p-value but show significant impact when removed from the data.
*(P-value < 0.1)

TABLE 2
Antibodies

| Antibody | Company, | Western | IHC | IHC Antigen retrieval |
|---|---|---|---|---|
| name | Catalog # | Dilution | Dilution | and buffer |
| ERG | Epitomics, | N/A | 1:150 | |
| | 2805-1 | | | |
| Syntenin | Abcam, | 1:1000 | 1:25 | Veg. Steamer 20 min |
| | ab62530 | | | with EDTA pH 9.0, plus |
| | | | | amplification |
| ING3 | Abcam, | 1:1000 | 1:100 | EDTA treated for 20 min |
| | ab10905 | | | as HIER |
| ANXA4 | Sigma, | 1:1000 | 1:100 | Retriever 2100 20 min |
| | HPA007393 | | | with Citrate pH 6.0 |
| FRP-3 | Santa Cruz | 1:500 | 1:25 | Retriever 2100 3 hr |
| (FRZB) | Biotechnology, | | | with EDTA pH 9.0 |
| | sc-13947 | | | |
| Ankyrin G | Santa Cruz | 1:1000 | 1:150 | Retriever 2100 3 hr |
| (ANK3) | Biotechnology, | | | with EDTA pH 9.0 |
| | sc-28561 | | | |
| MEIS2 | Abcam, | 1:1000 | 1:50 | Veg. Steamer 20 min |
| | ab49346 | | | with EDTA pH 9.0, plus |
| | | | | amplification |
| LEF1 | Abcam, | 1:5000 | 1:30 | Veg. Steamer 20 min |
| | ab22884 | | | with EDTA pH 9.0, plus |
| | | | | amplification |
| WNT2 | Epitomics, | 1:1000 | 1:40 | Retriever 2100 3 hr |
| | 3169-1 | | | with EDTA pH 9.0 |
| PLA2G7 | Abgent, | 1:1000 | 1:50 | Retriever 2100 3 hr |
| | AP9819c | | | with EDTA pH 9.0 |
| CHD5 | Abcam, | 1:1000 | 1:75 | Veg. Steamer10 min |
| | ab66516 | | | with EDTA pH 9.0 |
| ERG- | Santa Cruz | 1:5000 | N/A | N/A |
| 1/2/3 | Biotechnology, | | | |
| (C20) | sc-353 | | | |
| ING3 | Epitomics, | 1:1000 | N/A | N/A |
| | 3213-1 | | | |

TABLE 3
Real-time PCR primers used for relative expression analysis

| | | | |
|---|---|---|---|
| ANXA4 | Fwd: GGCAGGGACTTGATAGACGA | Syntenin | Fwd: TGGTGGCTCCTGTAACTGGT |
| | Rev: GCAGCTCTTGCACGTCATAC | | Rev: AGAGCTCCATCCTGCACAGT |
| WNT2 | Fwd: AGCTGAGCGCTTCTGCTCT | MEIS2 | Fwd: GTATGGGATCCGCTGTCAAC |
| | Rev: AGCTCTCATGTACCACCATGAA | | Rev: AAGGAGTCGGAGGAGCAGAC |
| PLA2G7 | Fwd: GGGCACCTTCTTGCGTTTAT | ERG | Fwd: TGGCTCAAGGAACTCTCCTG |
| | Rev: TCATTGAACCAAAGAGTAACCTCA | | Rev: ATAACTCTGCGCTCGTTCGT |
| ANK3 | Fwd: AAAGTCTGATGCCAATGCAA | TMPRSS2-1A | TAGGCGCGAGCTAAGCAGGAG |
| | Rev: GCATCCACATTGGCTTCTCT | ERG-4B | GTAGGCACACTCAAACAACGACTGG |
| FRZB | Fwd: TCCGGAAATAGGTCTTCTGTGT | GUSB | Fwd: TTGCTCACAAAGGTCACAGG |
| | Rev: CGGAGCTGATTTTCCTATGG | | Rev: CGTCCCACCTAGAATCTGCT |
| ING3 | Fwd: CGACAGCGAGTGACACAAAT | GAPDH | Fwd: CCCCACACACATGCACTTACC |
| | Rev: ATCCATTGCATTCTGCACCT | | Rev: CCTACTCCCAGGGCTTTGATT |
| LEF1 | Fwd: GACGAGATGATCCCCTTCAA | CHD5 | Fwd: CCGAGGAGATGGAGAATGAG |
| | Rev: AGGGCTCCTGAGAGGTTTGT | | Rev: TTCTTCCGCTTCCCTTTACA |

TABLE 4

| | Pro- | | | | ERG |
|---|---|---|---|---|---|
| | gres- | | | | asso- |
| | sion | BN | LPCA | CRPC | cia- |
| | P | (Mean | (Mean ± | (Mean ± | tion p |
| Markers | value | SD) | SD) | SD) | value |
| CHD5(cyto) | 0.000 | 1.28 ± 0.481 | 2.26 ± 0.569 | 2.69 ± 0.544 | 0.026 |
| Ankyrin(cyto) | 0.000 | 0.34 ± 0.509 | 1.52 ± 0.517 | 1.61 ± 0.502 | 0.203 |
| Ankyrin(mem) | 0.000 | 1.55 ± 0.724 | 2.09 ± 0.684 | 1.94 ± 0.725 | 0.010 |
| Syntenin(cyto) | 0.000 | 0.59 ± 0.523 | 2.04 ± 0.528 | 2.04 ± 0.713 | 0.378 |
| Meis2(cyto) | 0.000 | 1.22 ± 0.449 | 2.26 ± 0.516 | 2.23 ± 0.660 | 0.035 |
| FRP-3(cycto) | 0.000 | 1.07 ± 0.596 | 1.68 ± 0.592 | 2.53 ± 0.539 | 0.011 |
| LEF1(nuclear) | 0.000 | 1.26 ± 0.822 | 1.90 ± 0.640 | 1.00 ± 0.833 | 0.000 |
| PLA2G7(cyto) | 0.000 | 1.04 ± 0.630 | 1.79 ± 0.629 | 1.63 ± 0.809 | 0.000 |
| WNT2(cyto) | 0.000 | 0.58 ± 0.597 | 1.73 ± 0.610 | 1.96 ± 0.690 | 0.001 |
| ANX4(cyto) | 0.000 | 1.95 ± 0.590 | 1.60 ± 0.660 | 0.98 ± 0.887 | 0.083 |
| ING3(Abcam) | 0.000 | 1.59 ± 0.496 | 2.19 ± 0.549 | 2.17 ± 0.509 | 0.082 |

TABLE 5A
Univariate Analysis of 10 Gene Signature in Prostate Cancer (serum PSA up to 10 ng/ml)

| | Univariate | | | | Multivariate | | | |
|---|---|---|---|---|---|---|---|---|
| Data | p-value | HR | L-HR | H-HR | p-value | HR | L-HR | H-HR |
| Biopsy PSA* | 0.875563 | 0.929062 | 0.369897 | 2.333501 | 0.272958 | 0.569354 | 0.207988 | 1.448573 |
| Biopsy Gleason* | 0.022711 | 3.476986 | 1.190155 | 10.15786 | 0.017196 | 4.173129 | 1.288332 | 13.51748 |
| Surg. Margin+ | 0.208826 | 1.720481 | 0.738160 | 4.010047 | 0.593221 | 1.285584 | 0.511431 | 3.231569 |
| Path. Stage+ | 1.95E−06 | 3.97E+00 | 2.248342 | 6.995226 | 1.07E−05 | 4.625749 | 2.338845 | 9.148769 |
| Signature | 0.005214 | 3.095036 | 1.400874 | 6.838053 | 0.028335 | 2.568635 | 1.105264 | 5.969509 |

TABLE 5B
Univariate Analysis of 10 Gene Signature in Prostate Cancer (serum PSA up to 40 ng/ml)

| | Univariate | | | | Multivariate | | | |
|---|---|---|---|---|---|---|---|---|
| Data | p-value | HR | L-HR | H-HR | p-value | HR | L-HR | H-HR |
| Biopsy PSA* | 0.709127 | 1.182594 | 0.489933 | 2.854529 | 0.568555 | 0.760677 | 0.297048 | 1.947936 |
| Biopsy Gleason* | 0.000522 | 4.085652 | 1.844658 | 9.049135 | 0.052538 | 2.421902 | 0.990328 | 5.922898 |
| Surg. Margin+ | 0.060994 | 1.930817 | 0.970094 | 3.842983 | 0.813698 | 1.096955 | 0.508080 | 2.368349 |
| Path. Stage+ | 1.24E−06 | 3.33E+00 | 2.048358 | 5.418553 | 2.45E−04 | 2.874797 | 1.634987 | 5.054756 |
| Signature | 0.004407 | 2.658099 | 1.356204 | 5.209755 | 0.079012 | 1.927126 | 0.926791 | 4.007175 |

HR—average hazard ratio 95& CI
L-HR—low hazard ratio
H-HR—high hazard ratio
*—time of biopsy
+—time of surgery

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 3,817,837
U.S. Pat. No. 3,850,752
U.S. Pat. No. 3,939,350
U.S. Pat. No. 3,996,345
U.S. Pat. No. 4,275,149
U.S. Pat. No. 4,277,437
U.S. Pat. No. 4,366,241
U.S. Pat. No. 4,415,723
U.S. Pat. No. 4,458,066
U.S. Pat. No. 4,683,195
U.S. Pat. No. 4,683,202
U.S. Pat. No. 4,800,159
U.S. Pat. No. 4,883,750
U.S. Pat. No. 5,143,854
U.S. Pat. No. 5,202,231
U.S. Pat. No. 5,242,974
U.S. Pat. No. 5,279,721
U.S. Pat. No. 5,288,644
U.S. Pat. No. 5,324,633
U.S. Pat. No. 5,384,261
U.S. Pat. No. 5,405,783
U.S. Pat. No. 5,412,087
U.S. Pat. No. 5,424,186
U.S. Pat. No. 5,429,807
U.S. Pat. No. 5,432,049
U.S. Pat. No. 5,436,327
U.S. Pat. No. 5,445,934
U.S. Pat. No. 5,468,613
U.S. Pat. No. 5,470,710
U.S. Pat. No. 5,472,672
U.S. Pat. No. 5,492,806
U.S. Pat. No. 5,503,980
U.S. Pat. No. 5,510,270
U.S. Pat. No. 5,525,464
U.S. Pat. No. 5,527,681
U.S. Pat. No. 5,529,756
U.S. Pat. No. 5,532,128
U.S. Pat. No. 5,545,531
U.S. Pat. No. 5,547,839
U.S. Pat. No. 5,554,501
U.S. Pat. No. 5,556,752
U.S. Pat. No. 5,561,071
U.S. Pat. No. 5,571,639
U.S. Pat. No. 5,580,726
U.S. Pat. No. 5,580,732
U.S. Pat. No. 5,593,839
U.S. Pat. No. 5,599,672
U.S. Pat. No. 5,599,695
U.S. Pat. No. 5,610,287
U.S. Pat. No. 5,624,711
U.S. Pat. No. 5,631,134
U.S. Pat. No. 5,639,603
U.S. Pat. No. 5,654,413
U.S. Pat. No. 5,658,734
U.S. Pat. No. 5,661,028
U.S. Pat. No. 5,665,547
U.S. Pat. No. 5,667,972
U.S. Pat. No. 5,695,940
U.S. Pat. No. 5,700,637
U.S. Pat. No. 5,739,169
U.S. Pat. No. 5,744,305
U.S. Pat. No. 5,795,715
U.S. Pat. No. 5,800,992
U.S. Pat. No. 5,801,005
U.S. Pat. No. 5,807,522
U.S. Pat. No. 5,824,311
U.S. Pat. No. 5,830,645
U.S. Pat. No. 5,830,880
U.S. Pat. No. 5,837,196
U.S. Pat. No. 5,840,873
U.S. Pat. No. 5,843,640
U.S. Pat. No. 5,843,650
U.S. Pat. No. 5,843,651
U.S. Pat. No. 5,843,663
U.S. Pat. No. 5,846,708
U.S. Pat. No. 5,846,709
U.S. Pat. No. 5,846,717
U.S. Pat. No. 5,846,726
U.S. Pat. No. 5,846,729
U.S. Pat. No. 5,846,783
U.S. Pat. No. 5,846,945
U.S. Pat. No. 5,847,219
U.S. Pat. No. 5,849,481
U.S. Pat. No. 5,849,486
U.S. Pat. No. 5,849,487
U.S. Pat. No. 5,849,497
U.S. Pat. No. 5,849,546
U.S. Pat. No. 5,849,547
U.S. Pat. No. 5,851,772
U.S. Pat. No. 5,853,990
U.S. Pat. No. 5,853,992
U.S. Pat. No. 5,853,993
U.S. Pat. No. 5,856,092
U.S. Pat. No. 5,858,652
U.S. Pat. No. 5,861,244
U.S. Pat. No. 5,863,732
U.S. Pat. No. 5,863,753
U.S. Pat. No. 5,866,331
U.S. Pat. No. 5,866,366
U.S. Pat. No. 5,871,928
U.S. Pat. No. 5,876,932
U.S. Pat. No. 5,882,864
U.S. Pat. No. 5,889,136
U.S. Pat. No. 5,900,481
U.S. Pat. No. 5,905,024
U.S. Pat. No. 5,910,407
U.S. Pat. No. 5,912,124
U.S. Pat. No. 5,912,145
U.S. Pat. No. 5,912,148
U.S. Pat. No. 5,916,776
U.S. Pat. No. 5,916,779
U.S. Pat. No. 5,919,626
U.S. Pat. No. 5,919,630
U.S. Pat. No. 5,922,574
U.S. Pat. No. 5,925,517
U.S. Pat. No. 5,928,862
U.S. Pat. No. 5,928,869
U.S. Pat. No. 5,928,905
U.S. Pat. No. 5,928,906
U.S. Pat. No. 5,929,227
U.S. Pat. No. 5,932,413
U.S. Pat. No. 5,932,451
U.S. Pat. No. 5,935,791
U.S. Pat. No. 5,935,825
U.S. Pat. No. 5,939,291
U.S. Pat. No. 5,942,391
U.S. Pat. No. 6,004,755
U.S. Pat. No. 6,087,102
U.S. Pat. No. 6,368,799
U.S. Pat. No. 6,383,749
U.S. Pat. No. 6,506,559
U.S. Pat. No. 6,573,099
U.S. Pat. No. 6,617,112
U.S. Pat. No. 6,638,717
U.S. Pat. No. 6,720,138
U.S. Patent Publn. 2002/0168707
U.S. Patent Publn. 2003/0051263
U.S. Patent Publn. 2003/0055020
U.S. Patent Publn. 2003/0159161
U.S. Patent Publn. 2004/0064842
U.S. Patent Publn. 2004/0265839
U.S. Patent Publn. 2008/0009439
Abbondanzo et al., Breast Cancer Res. Treat., 16:182(151), 1990.

Allred et al., Arch. Surg., 125(1):107-113, 1990.
Attard et al., Cancer Res., 69:2912-2918, 2009.
Attard et al., Oncogene, 27:253-263, 2008.
Austin-Ward and Villaseca, Revista Medica de Chile, 126(7):838-845, 1998.
Barwick et al., Br. J. Cancer, 102:570-576, 2010.
Bellus, J. Macromol. Sci. Pure Appl. Chem., A31(1): 1355-1376, 1994.
Bismar et al., BJU Intl., 107:477-485, 2011.
Bosher and Labouesse, Nat. Cell. Biol., 2(2):E31-E36, 2000.
Brown et al. Immunol. Ser., 53:69-82, 1990.
Brummelkamp et al., Cancer Cell, 2:243-247, 2002.
Brummelkamp et al., Science, 296(5567):550-553, 2002.
Bukowski et al., Clinical Cancer Res., 4(10):2337-2347, 1998.
Bullinger et al., N Engl J. Med., 350:1605-1616, 2004.
Cai et al., Translational Oncology, 3:195-203, 2010.
Capaldi et al., Biochem. Biophys. Res. Comm., 74(2):425-433, 1977.
Carver et al., Nat. Genet., 41:619-624, 2009.
Christodoulides et al., Microbiology, 144(Pt 11):3027-3037, 1998.
Davidson et al., J. Immunother., 21(5):389-398, 1998.
De Jager et al., Semin. Nucl. Med., 23(2):165-179, 1993.
Demichelis et al., Oncogene, 26:4596-4599, 2007.
Doolittle and Ben-Zeev, Methods Mol, Biol, 109:215-237, 1999.
Esgueva et al., Mod. Pathol., 23:539-546, 2010.
European Appln. 320 308
European Appln. 329 822
European Appln. 373 203
European Appln. 785 280
European Appln. 799 897
Fire et al., Nature, 391(6669):806-811, 1998.
Fodor et al., Science, 251:767-773, 1991.
Frohman, In: PCR Protocols: A Guide To Methods And Applications, Academic Press, N.Y., 1990.
GB Application No. 2 202 328
Glinsky et al., J Clin. Invest., 113:913-23, 2004.
Grishok et al., Science, 287:2494-2497, 2000.
Gulbis and Galand, Hum. Pathol., 24(12):1271-1285, 1993.
Gupta et al., Cancer Res., 70(17):6735-6745, 2010.
Hacia et al., Nat. Genet., 14:441-449, 1996.
Haffner et al., Nat. Genet., 42(8):668-675, 2010.
Han et al., Mod. Pathol., 22:1083-1093, 2009a.
Han et al., Mod. Pathol., 22:1176-1185, 2009b.
Hanibuchi et al., Int. J. Cancer, 78(4):480-485, 1998.
Harlow and Lane, In: Antibodies: A Laboratory Manual, Cold Spring Harbor Laboratory, Cold Spring Harbor, N.Y., 346-348, 1988.
Hawksworth et al., Prostate Cancer Prost. Dis., 13:311-315, 2010.
Helgeson et al., Cancer Res., 68:73-80, 2008.
Hellstrand et al., Acta Oncologica, 37(4):347-353, 1998.
Hui and Hashimoto, Infection Immun., 66(11):5329-5336, 1998.
Innis et al., Proc. Natl. Acad. Sci. USA, 85(24):9436-9440, 1988.
Ju et al., Gene Ther., 7(19):1672-1679, 2000.
Ketting et al., Cell, 99(2):133-141, 1999.
King et al., Nat. Genet., 41:524-526, 2009.
Kunderfranco et al., PLoS ONE, 5:e10547, 2010.
Kwoh et al., Proc. Natl. Acad. Sci. USA, 86:1173, 1989.
Lee et al., Biochim. Biophys. Acta, 1582:175-177, 2002.
Leshem et al., PLoS ONE, 6:e21650, 2011.
Lin and Avery, Nature, 402:128-129, 1999.
MacBeath and Schreiber, Science, 289(5485):1760-1763, 2000.
Merico et al., PLoS ONE, 5:e13984, 2010.
Mitchell et al., Ann. NY Acad. Sci., 690:153-166, 1993.
Mitchell et al., J. Clin. Oncol., 8(5):856-869, 1990.
Montgomery et al., Proc. Natl. Acad. Sci. USA, 95:15502-15507, 1998.
Morton et al., Arch. Surg., 127:392-399, 1992.
Mosquera et al., J. Pathol., 212:91-101, 2007.
Nakamura et al., In: Handbook of Experimental Immunology (4^{th} Ed.), Weir et al. (Eds), 1:27, Blackwell Scientific Publ., Oxford, 1987.
Nam et al., Br. J. Cancer, 97:1690-1695, 2007.
Ohara et al., Proc. Natl. Acad. Sci. USA, 86:5673-5677, 1989.
Pandey and Mann, Nature, 405(6788):837-846, 2000.
Paul et al., Nature Biotechnol., 20:505-508, 2002.
Pawitan et al., Breast Cancer Res., 7:R953-964, 2005.
PCT Appln. PCT/US87/00880
PCT Appln. PCT/US89/01025
PCT Appln. WO 00/44914
PCT Appln. WO 01/36646
PCT Appln. WO 01/68836
PCT Appln. WO 01/38580
PCT Appln. WO 01/68255
PCT Appln. WO 03/020898
PCT Appln. WO 03/022421
PCT Appln. WO 03/023058
PCT Appln. WO 03/029485
PCT Appln. WO 03/040410
PCT Appln. WO 03/053586
PCT Appln. WO 03/066906
PCT Appln. WO 03/067217
PCT Appln. WO 03/076928
PCT Appln. WO 03/087297
PCT Appln. WO 03/091426
PCT Appln. WO 03/093810
PCT Appln. WO 03/100012
PCT Appln. WO 03/100448A1
PCT Appln. WO 04/020085
PCT Appln. WO 04/027093
PCT Appln. WO 09/923,256
PCT Appln. WO 09/936,760
PCT Appln. WO 88/10315
PCT Appln. WO 89/06700
PCT Appln. WO 90/07641
PCT Appln. WO 93/17126
PCT Appln. WO 95/11995
PCT Appln. WO 95/21265
PCT Appln. WO 95/21944
PCT Appln. WO 95/35505
PCT Appln. WO 96/31622
PCT Appln. WO 97/10365
PCT Appln. WO 97/27317
PCT Appln. WO 97/43450
PCT Appln. WO 99/32619
PCT Appln. WO 99/35505
Pease et al., Proc. Natl. Acad. Sci. USA, 91:5022-5026, 1994.
Pietras et al., Oncogene, 17(17):2235-2249, 1998.
Qin et al., Proc. Natl. Acad. Sci. USA, 95(24):14411-14416, 1998.
Ravindranath and Morton, Intern. Rev. Immunol., 7: 303-329, 1991.
Reid et al., Br. J. Cancer, 102:678-684, 2010.
Ribeiro et al., PLoS ONE, 6:e22317, 2011.
Rickman et al., Cancer Res., 69:2734-2738, 2009.
Rickman et al., Neoplasia, 12:1031-1040, 2010.
Ringner et al., PloS one, 6:317911, 2011.
Rosenberg et al., Ann. Surg. 210(4):474-548, 1989.
Rosenberg et al., N. Engl. J. Med., 319:1676, 1988.
Sambrook and Russell, Molecular Cloning: A Laboratory Manual 3^{rd} Ed., Cold Spring Harbor Laboratory Press, 2001.
Sharp and Zamore, Science, 287:2431-2433, 2000.
Sharp, Genes Dev., 13:139-141, 1999.
Shoemaker et al., Nature Genetics, 14:450-456, 1996.
Squire, Nat. Genet., 41:509-510, 2009.
Szklarczyk et al. Nucleic Acids Res., 39:D561-568, 2011.
Tabara et al., Cell, 99(2):123-132, 1999.
Taylor et al., Cancer Cell, 18:11-22, 2010.
Tomlins et al., Cancer Res., 66:3396-3400, 2006.
Tomlins et al., Sci. Transl. Med., 3(94):94ra72, 2011.
Tomlins et al., Science, 310:644-648, 2005.
UK Appln. 8 803 000
Vainio et al., Am. J. Pathol., 178:525-536, 2011.
Varambally et al., Cancer Cell, 8:393-406, 2005.
Varshaysky and Gottlieb, Bioinformatics, 22:e507-513, 2006.
Walker et al., Nucleic Acids Res. 20(7):1691-1696, 1992.
Wang et al., Cancer Res., 71(4):1325-1333, 2010.
Wang et al., Cancer Res., 71:1325-1333, 2011
Wincott et al., Nucleic Acids Res., 23(14):2677-2684, 1995.
Yoshimoto et al., Br. J. Cancer, 97:678-685, 2007.
Yoshimoto et al., Mod. Pathol., 21(12):1451-1460, 2008.
Yu et al., J. Am. Chem. Soc., 124(23):6576-6583, 2002.

1. A method of predicting prognosis in a human subject diagnosed with prostate, leukemia or breast cancer comprising obtaining expression information for four or more of the following genes in a cancer sample obtained from said subject:
ING3, LEF1, FRZB, ANXA4, MEIS2,
SDCBP, ANK3, CHD5, PLA2G7, and WNT2,
wherein a decrease in expression of ING3, LEF1, FRZB, MEIS2 and ANXA4 as compared to expression observed in non-cancer cells, and an increase in expression of SDCBP, ANK3, CHD5, PLA2G7 and WNT2, as compared to expression observed in non-cancer cells, indicates a poor prognosis.

2. The method of claim 1, wherein the decrease and/or increase of expression is at least 0.5-fold.

3. The method of claim 1, further comprising staging said prostate, leukemia or breast cancer based on said expression information.

4. The method of claim 1, further comprising obtaining information on PTEN expression or HER2 status.

5. The method of claim 1, wherein obtaining expression information comprises assessing protein expression.

6. (canceled)

7. The method of claim 1, wherein obtaining expression information expression comprises assessing mRNA expression or gene methylation status.

8. (canceled)

9. The method of claim 1, wherein said expression observed in said non-cancer cell is a pre-determined standard.

10. The method of claim 1, wherein said expression observed in said non-cancer cell is determined by assessing expression in a non-cancer cell from said subject.

11. The method of claim 1, further comprising obtaining said cancer sample.

12. The method of claim 11, wherein obtaining comprises taking a biopsy or blood sample from said subject.

13. The method of claim 1, wherein said prostate, leukemia or breast cancer is metastatic.

14. The method of claim 1, wherein said prostate, leukemia or breast cancer is localized.

15. The method of claim 1, wherein prognosis is length of survival.

16. The method of claim 15, wherein length of survival is disease-specific length of survival.

17. The method of claim 15, wherein length of survival is overall survival.

18. The method of claim 1, wherein prognosis is length of time to recurrence.

19. The method of claim 18, further comprising treating said patient with chemotherapy if said subject has a poor prognosis as compared to median.

20. The method of claim 18, further comprising not treating said subject with chemotherapy if said subject has a favorable prognosis as compared to median.

21. The method of claim 19, further comprising treating said subject with adjuvant chemotherapy.

22. The method of claim 2, wherein information on 5-9 markers is obtained.

23-26. (canceled)

27. The method of claim 1, wherein information on all 10 markers is obtained.

28-30. (canceled)

31. A method of predicting prognosis in a human subject diagnosed with breast cancer comprising obtaining expression information for the following genes in a cancer sample obtained from said subject:
ING3, LEF1, FRZB, and ANXA4,
wherein a decrease in expression of ING3, LEF1, FRZB, and ANXA4 as compared to expression observed in non-cancer cells, indicates a poor prognosis.

32-52. (canceled)
